SEQUENCE LISTING (34 sequences)

SEQ ID NO: 1 - protein, 26 aa, Artificial Sequence [sequence omitted]

SEQ ID NO: 2 - protein, 17 aa, Artificial Sequence
  Description of Artificial Sequence Synthetic peptide
LAWYQQKPGQAPRLLIY

SEQ ID NO: 3 - protein, 36 aa, Artificial Sequence [sequence omitted]

SEQ ID NO: 4 - protein, 10 aa, Artificial Sequence
  Description of Artificial Sequence Synthetic peptide
FGQGTKVEIK

SEQ ID NO: 5 - protein, 7 aa, Artificial Sequence
  Description of Artificial Sequence Synthetic peptide
QSVSSSY

SEQ ID NO: 6 - protein, 3 aa, Artificial Sequence
  Description of Artificial Sequence Synthetic peptide
GAS

SEQ ID NO: 7 - protein, 9 aa, Artificial Sequence
  Description of Artificial Sequence Synthetic peptide
QQYGSSPLT

SEQ ID NO: 8 - protein, 108 aa, Artificial Sequence
  Description of Artificial Sequence Synthetic polypeptide
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGQGTKVEIK

SEQ ID NO: 9 - protein, 215 aa, Artificial Sequence
  Description of Artificial Sequence Synthetic polypeptide
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGQGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 10 - DNA, 645 bp, Artificial Sequence
  Description of Artificial Sequence Synthetic polynucleotide
gaaattgtgctgacccagagcccgggcaccctgagcctgagcccgggcgaacgcgcgaccctgagctgccgcgcgagccagagcgtgagcagcagctatctggcgtggtatcagcagaaaccgggccaggcgccgcgcctgctgatttatggcgcgagcagccgcgcgaccggcattccggatcgctttagcggcagcggttccggcaccgattttaccctgaccattagccgcctggaaccggaagattttgcggtgtattattgccagcagtatggcagcagcccgctgacctttggccagggcaccaaagtggaaattaaacgggctgatgctgcaccgactgtgtccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgt

SEQ ID NO: 11 - protein, 25 aa, Artificial Sequence [sequence omitted]

SEQ ID NO: 12 - protein, 17 aa, Artificial Sequence
  Description of Artificial Sequence Synthetic peptide
ISWVRQAPGQGLEWMGG

SEQ ID NO: 13 - protein, 17 aa, Artificial Sequence
  Description of Artificial Sequence Synthetic peptide
ISWVRQAPGQGLEWMGY

SEQ ID NO: 14 - protein, 38 aa, Artificial Sequence
  Description of Artificial Sequence Synthetic polypeptide
EYAQKFQGRVTITADESTSTAYMELSSLRSEDTAVYYC

SEQ ID NO: 15 - protein, 38 aa, Artificial Sequence
  Description of Artificial Sequence Synthetic polypeptide
NYAQKFQGRVTITADESTSTAYMELSSLRSEDTAVYYC

SEQ ID NO: 16 - protein, 11 aa, Artificial Sequence
  Description of Artificial Sequence Synthetic peptide
WGQGTLVTVSS

SEQ ID NO: 17 - protein, 8 aa, Artificial Sequence
  Description of Artificial Sequence Synthetic peptide
GGTFKSDA

SEQ ID NO: 18 - protein, 8 aa, Artificial Sequence
  Description of Artificial Sequence Synthetic peptide
GGTFKSYA

SEQ ID NO: 19 - protein, 8 aa, Artificial Sequence
  Description of Artificial Sequence Synthetic peptide
IRPNEGNA

SEQ ID NO: 20 - protein, 8 aa, Artificial Sequence
  Description of Artificial Sequence Synthetic peptide
ISPESGTA

SEQ ID NO: 21 - protein, 17 aa, Artificial Sequence
  Description of Artificial Sequence Synthetic peptide
ARGRYGAYRLVYYAFDY

SEQ ID NO: 22 - protein, 19 aa, Artificial Sequence
  Description of Artificial Sequence Synthetic peptide
ARERYYYGYRRYRYYGMDV

SEQ ID NO: 23 - protein, 124 aa, Artificial Sequence
  Description of Artificial Sequence Synthetic polypeptide
QVQLVQSGAEVKKPGSSVKVSCKASGGTFKSDAISWVRQAPGQGLEWMGGIRPNEGNAEYAQKFQGRVTITADESTSTAYMELSSLRSEDTAVYYCARGRYGAYRLVYYAFDYWGQGTLVTVSS

SEQ ID NO: 24 - protein, 126 aa, Artificial Sequence
  Description of Artificial Sequence Synthetic polypeptide
QVQLVQSGAEVKKPGSSVKVSCKASGGTFKSYAISWVRQAPGQGLEWMGYISPESGTANYAQKFQGRVTITADESTSTAYMELSSLRSEDTAVYYCARERYYYGYRRYRYYGMDVWGQGTLVTVSS

SEQ ID NO: 25 - protein, 454 aa, Artificial Sequence
  Description of Artificial Sequence Synthetic polypeptide
QVQLVQSGAEVKKPGSSVKVSCKASGGTFKSDAISWVRQAPGQGLEWMGGIRPNEGNAEYAQKFQGRVTITADESTSTAYMELSSLRSEDTAVYYCARGRYGAYRLVYYAFDYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 26 - protein, 456 aa, Artificial Sequence
  Description of Artificial Sequence Synthetic polypeptide
QVQLVQSGAEVKKPGSSVKVSCKASGGTFKSYAISWVRQAPGQGLEWMGYISPESGTANYAQKFQGRVTITADESTSTAYMELSSLRSEDTAVYYCARERYYYGYRRYRYYGMDVWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 27 - DNA, 1362 bp, Artificial Sequence
  Description of Artificial Sequence Synthetic polynucleotide
caggtgcagctggtgcagagcggcgcggaagtgaaaaaaccgggcagcagcgtgaaagtgagctgcaaagcgagcggcggcaccttcaaatccgacgcgattagctgggtgcgccaggcgccgggccagggcctggaatggatgggcggtattcgcccaaacgaggggaatgctgagtacgcgcagaaatttcagggccgcgtgaccattaccgctgatgaaagcaccagcaccgcgtatatggaactgagcagcctgcgcagcgaagataccgcggtgtattattgcgcgcgtggtcgatatggtgcatatcgtctggtttactatgcgtttgactactggggccagggcaccctggtgaccgtctcgagtgccaaaacaacagcaccaagtgtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaa

SEQ ID NO: 28 - DNA, 1368 bp, Artificial Sequence
  Description of Artificial Sequence Synthetic polynucleotide
caggtgcagctggtgcagagcggcgcggaagtgaaaaaaccgggcagcagcgtgaaagtgagctgcaaagcgagcggcggcacctttaaatcctatgcgatttcctgggtgcgccaggcgccgggccagggcctggaatggatgggctatatttccccagagagtggcactgccaattatgcgcagaaatttcagggccgcgtgaccattaccgctgatgaaagcaccagcaccgcgtatatggaactgagcagcctgcgcagcgaagataccgcggtgtattattgcgcgcgtgaacgttactactatggctatcgtcgttaccggtattacggtatggatgtttggggccagggcaccctggtgaccgtctcgagtgccaaaacaacagcaccaagtgtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaa

SEQ ID NO: 29 - protein, 110 aa, Artificial Sequence
  Description of Artificial Sequence Synthetic polypeptide
QSALTQPPSVSEAPRQRVTISCSGSASNIGNNGVNWYQQLPGKTPKLLIYNDDLLPSGVSDRFSGSKSGTSASLAISGLQSEDEADYFCAAWDDSLNGLVFGGGTKLTVL

SEQ ID NO: 30 - protein, 119 aa, Artificial Sequence
  Description of Artificial Sequence Synthetic polypeptide
QVQLQQSGPGLVKPSQTLSLTCAISGDSVSSKSGAWNWIRQSPSRGLEWLGRTYYRSKWYNEYAVSVKSRITINPDTSKNQFSLQLNSVTPEDTAVYYCTRVDTDFDYWGQGTLVTVSS

SEQ ID NO: 31 - protein, 491 aa, Artificial Sequence
  Description of Artificial Sequence Synthetic polypeptide
MAWVWTLLFLMAAAQSIQAQSALTQPPSVSEAPRQRVTISCSGSASNIGNNGVNWYQQLPGKTPKLLIYNDDLLPSGVSDRFSGSKSGTSASLAISGLQSEDEADYFCAAWDDSLNGLVFGGGTKLTVLGGSEGKSSGSGSESKSTGGSQVQLQQSGPGLVKPSQTLSLTCAISGDSVSSKSGAWNWIRQSPSRGLEWLGRTYYRSKWYNEYAVSVKSRITINPDTSKNQFSLQLNSVTPEDTAVYYCTRVDTDFDYWGQGTLVTVSSTSTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 32 - protein, 491 aa, Artificial Sequence
  Description of Artificial Sequence Synthetic polypeptide
MAWVWTLLFLMAAAQSIQAQVQLQQSGPGLVKPSQTLSLTCAISGDSVSSKSGAWNWIRQSPSRGLEWLGRTYYRSKWYNEYAVSVKSRITINPDTSKNQFSLQLNSVTPEDTAVYYCTRVDTDFDYWGQGTLVTVSSGGSEGKSSGSGSESKSTGGSQSALTQPPSVSEAPRQRVTISCSGSASNIGNNGVNWYQQLPGKTPKLLIYNDDLLPSGVSDRFSGSKSGTSASLAISGLQSEDEADYFCAAWDDSLNGLVFGGGTKLTVLTSTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 33 - protein, 229 aa, Homo sapiens
MARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLDKDDSKAGMEEDHTYEGLDIDQTATYEDIVTLRTGEVKWSVGEHPGQE

SEQ ID NO: 34 - protein, 19 aa, Artificial Sequence
  Description of Artificial Sequence Synthetic peptide
MAWVWTLLFLMAAAQSIQA

(19) United States
(12) Patent Application Publication
(10) Pub. No.: US 2023/0094867 A1
(43) Pub. Date: Mar. 30, 2023

(54) ANTI-IDIOTYPIC ANTIBODIES AGAINST ANTI-CD79B ANTIBODIES

(71) Applicant: Janssen Biotech, Inc., Horsham, PA (US)

(72) Inventors: Katharine D. Grugan, Havertown, PA (US); Christian R. Martinez, San Diego, CA (US); Nan Wei, Blue Bell, PA (US); Hong Mimi Zhou, San Diego, CA (US)

(21) Appl. No.: 17/825,244

(22) Filed: May 26, 2022

Related U.S. Application Data
(60) Provisional application No. 63/195,248, filed on Jun. 1, 2021.

(51) Int. Cl. C07K 16/42; G01N 33/68

(52) U.S. Cl. CPC C07K 16/4258; G01N 33/686; C07K 2317/622; C07K 2317/55; C07K 2317/92; G01N 2333/7051

(57) ABSTRACT

In certain aspects, the disclosure relates to anti-idiotype antibodies and antigen-binding portions thereof that specifically bind a CD9B441 containing protein, e.g., an antibody or antigen-binding portions thereof. In some aspects, the anti-idiotype antibodies and antigen-binding portions of the present disclosure can be used in methods to detect and quantify cells expressing chimeric antigen receptors that include CD9B441.

### CROSS-REFERENCE TO RELATED APPLICATIONS

This application claims the benefit of U.S. Provisional Application Ser. No. 63/195,248, filed 1 Jun. 2021. The entire content of the aforementioned application is incorporated herein by reference in its entirety.

### SEQUENCE LISTING

The instant application contains a Sequence Listing which has been submitted electronically in ASCII format and is hereby incorporated by reference in its entirety. Said ASCII copy, created on May 12, 2022, is named JBI6565USNP1_SL.txt and is 37,579 bytes in size.

### TECHNICAL FIELD

The invention relates to anti-idiotype antibodies and antigen-binding portions thereof that specifically bind a CD9B441 containing protein, e.g., an antibody or antigen-binding portions thereof. Methods to detect and quantify cells expressing chimeric antigen receptors that include CD9B441 are also provided.

### BACKGROUND

Recent advances in the understanding of the delivery of genomic material and integration into a target's genome have great potential to transform the standard-of-care for a variety of diseases. T cell therapy utilizes isolated T cells that have been genetically modified to enhance their specificity for a specific tumor associated antigen. Genetic modification may involve the expression of a chimeric antigen receptor (CAR) or an exogenous T cell receptor to provide new antigen specificity onto the T cell. T cells expressing chimeric antigen receptors (CAR-T cells) can induce tumor immunoreactivity.

One particular CAR target of interest is CD79b. To detect the antigen or pathogen, B cells have B cell receptors (BCRs) on the cell surface, which is a multicomponent receptor composed of a transmembrane immunoglobulin molecule (mIg) and a disulfide linked heterodimer of CD79a (Igα) and CD79b (Igβ). CD79b is highly expressed in a wide range of B-cell lymphomas. Its expression has been shown to be critical for cancer cell viability of most diffuse large B-cell lymphoma (DLBCL) tumor models. Therefore, the development of resistance to CD79b targeted agents through antigen loss may be less likely, making it an attractive target for the development of novel immunotherapeutic approaches. In the clinic, Polatuzumab (Polivy™), an antibody-drug conjugate (ADC) molecule targeting CD79b, has recently been approved for the treatment of relapsed/refractory (r/r) DLBCL (See, e.g., Polson et al., Blood, 110: 616-623 (2007)). Polatuzumab treatments results in an increase in complete response (CR) and duration of response (DOR) rates when combined with standard of care treatments (bendamustine and Rituximab), validating CD79b as a valuable clinical target (See, e.g., Palanca-Wessels et al., The Lancet Oncology, 16(6): 704-715 (2015)). Accordingly, there is a need for developing CD79b-targeting CAR-T therapy. There is also a need for anti-idiotype antibodies directed to such CARs in order to detect, purify, or select proteins and cells expressing the CAR.

### SUMMARY OF THE INVENTION

The disclosure provides anti-idiotype antibodies and antigen-binding portions thereof that specifically bind a CD9B441 containing protein, e.g., an antibody or antigen-binding portions thereof. The disclosure also provides nucleic acids encoding the anti-idiotype antibodies and antigen-binding portions thereof, methods of producing the anti-idiotype antibodies and antigen-binding portions thereof, methods of detecting CD9B441 using the anti-idiotype antibodies and antigen-binding portions thereof and kits including the anti-idiotype antibodies and antigen-binding portions thereof.

In one aspect, the disclosure provides an anti-idiotype antibody or antigen-binding portion thereof that specifically binds an anti-CD79b antibody, such as a target antibody that comprises CD9B441. In some embodiments, the target antibody or antigen-binding portion thereof comprises a light chain variable (VL) domain comprising the amino acid sequence of SEQ ID NO: 29 and a heavy chain variable (VH) domain comprising the amino acid sequence of SEQ ID NO: 30.

In other embodiments, the anti-idiotype antibody or antigen-binding portion is for use in detecting CD9B441 in a biologic sample comprising: (a) providing a biological sample; (b) contacting the biological sample with the anti-idiotype antibody or antigen-binding portion; and (c) detecting the anti-idiotype antibody or antigen-binding portion.

In another aspect, the disclosure provides an anti-idiotype antibody or antigen-binding portion thereof that specifically binds CD9B441, wherein the anti-idiotype antibody or antigen-binding portion comprises a complementarity determining region of a light chain variable (VL) domain comprising, the LCDR1 having the amino acid sequence of SEQ ID NO: 5, the LCDR2 having the amino acid sequence of SEQ ID NO: 6, the LCDR3 having the amino acid sequence of SEQ ID NO: 7; and further comprises a complementarity determining region of a heavy chain variable (VH) domain comprising HCDR1-3 having amino acid sequences selected from the group consisting of an HCDR1 having the amino acid sequence of SEQ ID NO: 17, an HCDR2 having the amino acid sequence of SEQ ID NO: 19, an HCDR3 having the amino acid sequence of SEQ ID NO: 21; and an HCDR1 having the amino acid sequence of SEQ ID NO: 18, an HCDR2 having the amino acid sequence of SEQ ID NO: 20, and an HCDR3 having the amino acid sequence of SEQ ID NO: 22.

In some embodiments, the anti-idiotype antibody or antigen-binding portion thereof comprises a VL domain that has an amino acid sequence having at least 90% sequence identity to SEQ ID NO: 8 and a VH domain that has an amino acid sequence having at least 90% sequence identity to SEQ ID NO: 23. In some embodiments, the anti-idiotype antibody or antigen-binding portion thereof comprises a light chain that has an amino acid sequence having at least 90% sequence identity to SEQ ID NO: 9 and a heavy chain that has an amino acid sequence having at least 90% identity to SEQ ID NO: 25. In some embodiments, the anti-idiotype antibody or antigen-binding portion thereof comprises a VL domain that has an amino acid sequence having at least about 80%, 81%, 82%, 83%, 84%, 85%, 86%, 87%, 88%, 89%, 90%, 91%, 92%, 93%, 94%, 95%, 96%, 97%, 98% or 99% sequence identity to SEQ ID NO: 8. In some embodiments, the anti-idiotype antibody or antigen-binding portion thereof comprises a VH domain that has an amino acid sequence having at least about 80%, 81%, 82%, 83%, 84%, 85%, 86%, 87%, 88%, 89%, 90%, 91%, 92%, 93%, 94%, 95%, 96%, 97%, 98% or 99% sequence identity to SEQ ID NO: 23.

In certain embodiments, the anti-idiotype antibody or antigen-binding portion thereof comprises the VL domain of SEQ ID NO: 8 and the VH domain of SEQ ID NO: 23. In certain embodiments, the anti-idiotype antibody or antigen-binding portion thereof comprises a light chain comprising the amino acid sequence of SEQ ID NO: 9 and further comprises a heavy chain comprising the amino acid sequence of SEQ ID NO: 25.

In some embodiments, the anti-idiotype antibody or antigen-binding portion thereof comprises a VL domain that has an amino acid sequence having at least 90% sequence identity to SEQ ID NO: 8 and a VH domain that has an amino acid sequence having at least 90% sequence identity to SEQ ID NO: 24. In some embodiments, the anti-idiotype antibody or antigen-binding portion thereof comprises a light chain that has an amino acid sequence having at least 90% sequence identity to SEQ ID NO: 9 and a heavy chain that has an amino acid sequence having at least 90% identity to SEQ ID NO: 26. In some embodiments, the anti-idiotype antibody or antigen-binding portion thereof comprises a VL domain that has an amino acid sequence having at least about 80%, 81%, 82%, 83%, 84%, 85%, 86%, 87%, 88%, 89%, 90%, 91%, 92%, 93%, 94%, 95%, 96%, 97%, 98% or 99% sequence identity to SEQ ID NO: 8. In some embodiments, the anti-idiotype antibody or antigen-binding portion thereof comprises a VH domain that has an amino acid sequence having at least about 80%, 81%, 82%, 83%, 84%, 85%, 86%, 87%, 88%, 89%, 90%, 91%, 92%, 93%, 94%, 95%, 96%, 97%, 98% or 99% sequence identity to SEQ ID NO: 24.

In certain embodiments, the anti-idiotype antibody or antigen-binding portion thereof comprises the VL domain of SEQ ID NO: 8 and the VH domain of SEQ ID NO: 24. In certain embodiments, the anti-idiotype antibody or antigen-binding portion thereof comprises a light chain comprising the amino acid sequence of SEQ ID NO: 9 and further comprises a heavy chain comprising the amino acid sequence of SEQ ID NO: 26.

In some embodiments, the antigen-binding portion is selected from a Fab, F(ab′)2, or scFv. In some embodiments, the antibody is a monoclonal antibody. In some embodiments, the antibody is a chimeric antibody. In some embodiments, the chimeric antibody comprises a murine IgG2a framework. In some other embodiments, the antibody is a fully human antibody. In some embodiments, the anti-idiotype antibody or antigen-binding portion thereof is specific to CD9B441, wherein CD9B441 is within the antigen-binding domain of the extracellular portion of a chimeric antigen receptor (CAR). In some embodiments, CD9B441 is an scFv and the anti-idiotype antibody or antigen-binding portion specifically binds an epitope in the scFv of the CAR. In some embodiments, CD9B441 specifically binds CD79b. In some embodiments, the antibody or antigen-binding portion does not cross-react to other CD79b antibodies or other CD79b binding CARs. In some embodiments, the CAR has an amino acid sequence selected from the group consisting of SEQ ID NOs: 31-32.

In some embodiments, the disclosure provides a nucleic acid encoding the heavy chain, the light chain, or both, of the anti-idiotype antibody or antigen-binding portion.

In another aspect, the disclosure provides a nucleic acid encoding the heavy chain, the light chain, or both, of an anti-idiotype antibody or an antigen-binding portion thereof that specifically binds CD9B441, wherein said nucleic acid comprises: the nucleotide sequence of SEQ ID NO: 10; the nucleotide sequence of SEQ ID NO: 27; or both. In some embodiments, the disclosure provides a nucleic acid encoding the heavy chain, the light chain, or both, of an anti-idiotype antibody or an antigen-binding portion thereof that specifically binds CD9B441, wherein said nucleic acid comprises: the nucleotide sequence of SEQ ID NO: 10; the nucleotide sequence of SEQ ID NO: 28; or both. In another aspect, the disclosure provides a vector comprising the nucleic acid sequence. For example, the vector may be a self-replicating nucleic acid structure, or incorporated into the genome of a host cell into which it has been introduced. In some embodiments, the vector is an expression vector. In another aspect, the disclosure provides a host cell comprising the vector. In some embodiments, the host cell is a mammalian cell.

In another aspect, the disclosure provides a method of producing an anti-idiotype antibody or antigen-binding portion thereof that specifically binds CD9B441, said method comprising culturing a host cell under conditions that allow said antibody or antigen-binding portion to be expressed, wherein the host cell comprises nucleotide sequences coding the heavy chain and light chain of the antibody or antigen-binding portion, and isolating said antibody or antigen-binding portion from the culture. In some embodiments, the host cell encodes a vector comprising a nucleic acid encoding the anti-idiotype antibody or antigen-binding portion thereof.

In another aspect, the disclosure provides a method for detecting CD9B441 in a biologic sample comprising: (a) providing a biological sample; (b) contacting the biological sample with the anti-idiotype antibody or antigen-binding portion; and (c) detecting the anti-idiotype antibody or antigen-binding portion.

In another aspect, the disclosure provides a method for detecting expression of a chimeric antigen receptor (CAR) comprising CD9B441 in a biologic sample comprising: (a) providing a biological sample; (b) contacting the biological sample with the anti-idiotype antibody or antigen-binding portion; and (c) detecting the anti-idiotype antibody or antigen-binding portion, and thereby detecting the expression of the CAR.

In some embodiments, the antibody comprises a detectable label. In some embodiments, the method further comprises further comprises contacting the anti-idiotype antibody or antigen-binding portion with a detectable label before detecting the anti-idiotype antibody or antigen-binding portion. In some embodiments, the biological sample is blood, serum or urine.

In some aspects, the disclosure provides a kit for detecting CD9B441 in a biologic sample comprising: (a) an anti-idiotype antibody or antigen-binding portion; and (b) instructions for detecting the anti-idiotype antibody or antigen-binding portion.

In other aspects, the disclosure provides a method of purifying CD9B441 from a sample comprising: (a) providing a biological sample comprising CD9B441; (b) contacting the biological sample with the anti-idiotype antibody or antigen-binding portion; and (c) capturing the anti-idiotype antibody or antigen-binding portion, and thereby purifying CD9B441.

In other aspects, the disclosure provides a method of selecting CAR-T cells from a cell population comprising: (a) providing a biological sample comprising CAR-T cells; (b) contacting the biological sample with an anti-idiotype antibody or antigen-binding portion; and (c) capturing the anti-idiotype antibody or antigen-binding portion, and thereby selecting CAR-T cells. In some embodiments, the anti-idiotype antibody or antigen-binding portion thereof is specific to CD9B441.

The disclosure contemplates all combinations of any of the foregoing aspects and embodiments, as well as combinations with any of the embodiments set forth in the detailed description and examples.

### BRIEF DESCRIPTION OF THE DRAWINGS

For the purpose of illustrating the invention, there are depicted in the drawings certain embodiments of the disclosure. However, the disclosure is not limited to the precise arrangements and instrumentalities of the embodiments depicted in the drawings.

FIG. 1 shows a graphical representation of CD9B503-specific binding enrichment after round three panning detected with polyclonal ELISA.

FIG. 2 shows a graphical representation of the results of monoclonal Fab binding screening from CD9B503 target binding assays compared with negative control scFv fusion protein counter screening reagent CD9B504.

FIGS. 3A-3B show a dose dependent binding to CD9B441-HL SupT1 cells. FIG. 3A shows a dose dependent binding of A003B192 and A003B274 to CD9B441-HL SupT1 cells. No binding was detected in the presence of excess Fc-CD9B441-HL CAR fusion protein. FIG. 3B shows a graphical representation of the dose dependent binding of recombinant phycoerythrin-conjugated A003B192 to CD9B441-HL-SupT1 cells.

### DETAILED DESCRIPTION OF THE INVENTION

### Overview

The disclosure provides anti-idiotype antibodies and antigen-binding portions thereof that specifically bind a CD9B441 containing protein, e.g., an antibody or antigen-binding portions thereof. The anti-idiotype antibodies and antigen-binding portions of the present disclosure can be used in methods to detect and quantify cells expressing CARs that include CD9B441. Such methods may allow a researcher to determine whether a given batch of in vitro generated CAR-T cells have expressed the desired CAR and thus whether the cells are therapeutically useful for targeting the desired proteins. In the present disclosure, the anti-idiotype antibodies and antigen-binding portions target CD9B441, which itself targets CD79b, a protein associated with cancers including B-cell lymphoma.

### Definitions

As used in this specification and the appended claims, the singular forms “a,” “an,” and “the” include plural referents unless the content clearly dictates otherwise. Thus, for example, reference to “a cell” includes a combination of two or more cells, and the like.

The transitional terms “comprising,” “consisting essentially of,” and “consisting of” are intended to connote their generally accepted meanings in the patent vernacular; that is, (i) “comprising,” which is synonymous with “including,” “containing,” or “characterized by,” is inclusive or open-ended and does not exclude additional, unrecited elements or method steps; (ii) “consisting of” excludes any element, step, or ingredient not specified in the claim; and (iii) “consisting essentially of” limits the scope of a claim to the specified materials or steps “and those that do not materially affect the basic and novel characteristic(s)” of the claimed invention. Embodiments described in terms of the phrase “comprising” (or its equivalents) also provide as embodiments those independently described in terms of “consisting of” and “consisting essentially of.”

“Activation” or “stimulation” or “activated” or “stimulated” refers to induction of a change in the biologic state of a cell resulting in expression of activation markers, cytokine production, proliferation or mediating cytotoxicity of target cells. Cells may be activated by primary stimulatory signals. Co-stimulatory signals can amplify the magnitude of the primary signals and suppress cell death following initial stimulation resulting in a more durable activation state and thus a higher cytotoxic capacity. A “co-stimulatory signal” refers to a signal, which in combination with a primary signal, such as TCR/CD3 ligation, leads to T cell and/or NK cell proliferation and/or upregulation or downregulation of key molecules.

“Anti-idiotype antibody” or “anti-idiotypic antibody” refers to an antibody that specifically binds to the variable region of another antibody. In the case of A003B192 and A003B274, an anti-idiotype antibody specifically binds an anti-CD79b antibody.

“Antigen-binding portion,” “antigen-binding fragment” or “antigen-binding domain” refers to a portion of the protein that binds an antigen. Antigen binding fragments may be synthetic, enzymatically obtainable or genetically engineered polypeptides and include portions of an immunoglobulin that bind an antigen, such as the VH, the VL, the VH and the VL, Fab, Fab′, F(ab′)₂, Fd and Fv fragments, domain antibodies (dAb) consisting of one VH domain or one VL domain, shark variable IgNAR domains, camelized VH domains, VHH domains, minimal recognition units consisting of the amino acid residues that mimic the CDRs of an antibody, such as FR3-CDR3-FR4 portions, the HCDR1, the HCDR2 and/or the HCDR3 and the LCDR1, the LCDR2 and/or the LCDR3, alternative scaffolds that bind an antigen, and multispecific proteins comprising the antigen binding fragments. Antigen binding fragments (such as VH and VL) may be linked together via a synthetic linker to form various types of single antibody designs where the VH/VL domains may pair intramolecularly, or intermolecularly in those cases when the VH and VL domains are expressed by separate single chains, to form a monovalent antigen binding domain, such as single chain Fv (scFv) or diabody. Antigen binding fragments may also be conjugated to other antibodies, proteins, antigen binding fragments or alternative scaffolds which may be monospecific or multispecific to engineer bispecific and multispecific proteins.

“Cancer” refers to a broad group of various diseases characterized by the uncontrolled growth of abnormal cells in the body. Unregulated cell division and growth results in the formation of malignant tumors that invade neighboring tissues and may also metastasize to distant parts of the body through the lymphatic system or bloodstream. A “cancer” or “cancer tissue” can include a tumor.

“Full length antibody” is comprised of two heavy chains (HC) and two light chains (LC) inter-connected by disulfide bonds as well as multimers thereof (e.g. IgM). Each heavy chain is comprised of a heavy chain variable domain (VH) and a heavy chain constant domain, the heavy chain constant domain comprised of subdomains CH1, hinge, CH2 and CH3. Each light chain is comprised of a light chain variable domain (VL) and a light chain constant domain (CL). The VH and the VL may be further subdivided into regions of hypervariability, termed complementarity determining regions (CDR), interspersed with framework regions (FW). Each VH and VL is composed of three CDRs and four FW segments, arranged from amino-to-carboxy-terminus in the following order: FW1, CDR1, FW2, CDR2, FW3, CDR3 and FW4.

“Complementarity determining regions (CDR)” are antigen-binding sites in an antibody. CDRs may be defined using various terms: (i) Complementarity Determining Regions (CDRs), three in the VH (HCDR1, HCDR2, HCDR3) and three in the VL (LCDR1, LCDR2, LCDR3) are based on sequence variability (Wu and Kabat, J. Exp. Med. 132:211-50, 1970; Kabat et al., Sequences of Proteins of Immunological Interest, 5th Ed. Public Health Service, National Institutes of Health, Bethesda, Md., 1991). (ii) “Hypervariable regions”, “HVR”, or “HV”, three in the VH (H1, H2, H3) and three in the VL (L1, L2, L3) refer to the regions of an antibody variable domains which are hypervariable in structure as defined by Chothia and Lesk (Chothia and Lesk, Mol. Biol. 196:901-17, 1987). The International ImMunoGeneTics (IMGT) database (http://www_imgt_org) provides a standardized numbering and definition of antigen-binding sites. The correspondence between CDRs, HVs and IMGT delineations is described in Lefranc et al., Dev. Comparat. Immunol. 27:55-77, 2003. The term “CDR”, “HCDR1”, “HCDR2”, “HCDR3”, “LCDR1”, “LCDR2” and “LCDR3” as used herein includes CDRs defined by any of the methods described supra, Kabat, Chothia or IMGT, unless otherwise explicitly stated in the specification. The framework regions (FW) are adjacent to and between the CDRs in both the VL (LFW1, LFW2, LFW3, LFW4) and VH (HFW1, HFW2, HFW3, HFW4).

“Human antibody” refers to an antibody that is optimized to have minimal immune response when administered to a human subject. Variable regions of human antibody are derived from human immunoglobulin sequences. If human antibody contains a constant region or a portion of the constant region, the constant region is also derived from human immunoglobulin sequences. Human antibody comprises heavy and light chain variable regions that are “derived from” sequences of human origin if the variable regions of the human antibody are obtained from a system that uses human germline immunoglobulin or rearranged immunoglobulin genes. Such exemplary systems are human immunoglobulin gene libraries displayed on phage, and transgenic non-human animals such as mice or rats carrying human immunoglobulin loci. “Human antibody” typically contains amino acid differences when compared to the immunoglobulins expressed in humans due to differences between the systems used to obtain the human antibody and human immunoglobulin loci, introduction of somatic mutations or intentional introduction of substitutions into the frameworks or CDRs, or both. Typically, “human antibody” is at least about 80%, 81%, 82%, 83%, 84%, 85%, 86%, 87%, 88%, 89%, 90%, 91%, 92%, 93%, 94%, 95%, 96%, 97%, 98% or 99% identical in amino acid sequence to an amino acid sequence encoded by human germline immunoglobulin or rearranged immunoglobulin genes. In some cases, “human antibody” may contain consensus framework sequences derived from human framework sequence analyses, for example as described in Knappik et al., (2000) J. Mol. Biol. 296:57-86, or a synthetic HCDR3 incorporated into human immunoglobulin gene libraries displayed on phage, for example as described in Shi et al., (2010) J. Mol. Biol. 397:385-96, and in Int. Patent Publ. No. WO2009/085462. Antibodies in which at least one CDR is derived from a non-human species are not included in the definition of “human antibody”.

“Humanized antibody” refers to an antibody in which at least one CDR is derived from non-human species and at least one framework is derived from human immunoglobulin sequences. Humanized antibody may include substitutions in the frameworks so that the frameworks may not be exact copies of expressed human immunoglobulin or human immunoglobulin germline gene sequences.

“Isolated” refers to a homogenous population of molecules (such as synthetic polynucleotides or polypeptides) which have been substantially separated and/or purified away from other components of the system the molecules are produced in, such as a recombinant cell, as well as a protein that has been subjected to at least one purification or isolation step. “Isolated” refers to a molecule that is substantially free of other cellular material and/or chemicals and encompasses molecules that are isolated to a higher purity, such as to 80%, 81%, 82%, 83%, 84%, 85%, 86%, 87%, 88%, 89%, 90%, 91%, 92%, 93%, 94%, 95%, 96%, 97%, 98%, 99% or 100% purity.

“Modulate” refers to either enhanced or decreased ability of a test molecule to mediate an enhanced or a reduced response (i.e., downstream effect) when compared to the response mediated by a control or a vehicle.

“Natural killer cell” and “NK cell” are used interchangeably and synonymously herein. NK cell refers to a differentiated lymphocyte with a CD16+ CD56+ and/or CD57+ TCR− phenotype. NK cells are characterized by their ability to bind to and kill cells that fail to express “self” MHC/HLA antigens by the activation of specific cytolytic enzymes, the ability to kill tumor cells or other diseased cells that express a ligand for NK activating receptors, and the ability to release protein molecules called cytokines that stimulate or inhibit the immune response.

“Specifically binds,” “specific binding,” “specifically binding” or “binds” refer to a proteinaceous molecule binding to an antigen or an epitope within the antigen with greater affinity than for other antigens. Typically, the proteinaceous molecule binds to the antigen or the epitope within the antigen with an equilibrium dissociation constant (KD) of about 1×10⁻⁷ M or less, for example about 5×10⁻⁸ M or less, about 1×10⁻⁸ M or less, about 1×10⁻⁹ M or less, about 1×10⁻¹⁰ M or less, about 1×10⁻¹¹ M or less, or about 1×10⁻¹² M, 1×10⁻¹³ M, 1×10⁻¹⁴ M, 1×10⁻¹⁵ M or less, typically with the KD that is at least one hundred fold less than its KD for binding to a non-specific antigen (e.g., BSA, casein). In the context of the prostate neoantigens described here, “specific binding” refers to binding of the proteinaceous molecule to the prostate neoantigen without detectable binding to a wild-type protein the neoantigen is a variant of.

“Tumor cell” or a “cancer cell” refers to a cancerous, pre-cancerous or transformed cell, either in vivo, ex vivo, or in tissue culture, that has spontaneous or induced phenotypic changes. These changes do not necessarily involve the uptake of new genetic material. Although transformation may arise from infection with a transforming virus and incorporation of new genomic nucleic acid, uptake of exogenous nucleic acid or it can also arise spontaneously or following exposure to a carcinogen, thereby mutating an endogenous gene. Transformation/cancer is exemplified by morphological changes, immortalization of cells, aberrant growth control, foci formation, proliferation, malignancy, modulation of tumor specific marker levels, invasiveness, tumor growth in suitable animal hosts such as nude mice, and the like, in vitro, in vivo, and ex vivo.

The term “chimeric antigen receptor” or “CAR” as used herein is defined as a cell-surface receptor comprising an extracellular target-binding domain, a transmembrane domain and an intracellular signaling domain, all in a combination that is not naturally found together on a single protein. This particularly includes receptors wherein the extracellular domain and the intracellular signaling domain are not naturally found together on a single receptor protein. The chimeric antigen receptors of the present invention are intended primarily for use with lymphocyte such as T cells and natural killer (NK) cells.

The terms “T cell” and “T lymphocyte” are interchangeable and used synonymously herein. As used herein, T cell includes thymocytes, naive T lymphocytes, immature T lymphocytes, mature T lymphocytes, resting T lymphocytes, or activated T lymphocytes. A T cell can be a T helper (Th) cell, for example a T helper 1 (Th1) or a T helper 2 (Th2) cell. The T cell can be a helper T cell (HTL; CD4+ T cell) CD4+ T cell, a cytotoxic T cell (CTL; CD8+ T cell), a tumor infiltrating cytotoxic T cell (TIL; CD8+ T cell), CD4+CD8+ T cell, or any other subset of T cells. Other illustrative populations of T cells suitable for use in particular embodiments include naive T cells and memory T cells. Also included are “NKT cells”, which refer to a specialized population of T cells that express a semi-invariant T-cell receptor, but also express a variety of molecular markers that are typically associated with NK cells, such as NK1.1. NKT cells include NK1.1+ and NK1.1−, as well as CD4+, CD4−, CD8+ and CD8− cells. The TCR on NKT cells is unique in that it recognizes glycolipid antigens presented by the MHC I-like molecule CD1d. NKT cells can have either protective or deleterious effects due to their abilities to produce cytokines that promote either inflammation or immune tolerance. Also included are “gamma-delta T cells (γδ T cells),” which refer to a specialized population that to a small subset of T cells possessing a distinct TCR on their surface, and unlike the majority of T cells in which the TCR is composed of two glycoprotein chains designated α- and β-TCR chains, the TCR in γδ T cells is made up of a γ-chain and a δ-chain. γδ T cells can play a role in immunosurveillance and immunoregulation, and were found to be an important source of IL-17 and to induce robust CD8+ cytotoxic T cell response. Also included are “regulatory T cells” or “Tregs”, which refer to T cells that suppress an abnormal or excessive immune response and play a role in immune tolerance. Tregs are typically transcription factor Foxp3-positive CD4+ T cells and can also include transcription factor Foxp3-negative regulatory T cells that are IL producing CD4+ T cells.

As used herein, the term “antigen” refers to any agent (e.g., protein, peptide, polysaccharide, glycoprotein, glycolipid, nucleic acid, portions thereof, or combinations thereof) molecule capable of being bound by a T-cell receptor. An antigen is also able to provoke an immune response. An example of an immune response may involve, without limitation, antibody production, or the activation of specific immunologically competent cells, or both. A skilled artisan will understand that an antigen need not be encoded by a “gene” at all. It is readily apparent that an antigen can be generated synthesized or can be derived from a biological sample, or might be macromolecule besides a polypeptide. Such a biological sample can include, but is not limited to a tissue sample, a tumor sample, a cell or a fluid with other biological components, organisms, subunits of proteins/antigens, killed or inactivated whole cells or lysates.

The terms “antibody” and “antibodies” refer to monoclonal antibodies, multispecific antibodies, human antibodies, humanized antibodies, chimeric antibodies, single-chain Fvs (scFv), single chain antibodies, Fab fragments, F(ab′) fragments, disulfide-linked Fvs (sdFv), intrabodies, minibodies, diabodies and anti-idiotype (anti-Id) antibodies (including, e.g., anti-Id antibodies to antigen specific TCR), and epitope-binding fragments of any of the above. The terms “antibody” and “antibodies” also refer to covalent diabodies such as those disclosed in U.S. Pat. Appl. Pub. 2007/0004909 and Ig-DARTS such as those disclosed in U.S. Pat. Appl. Pub. 2009/0060910. Antibodies useful as a TCR-binding molecule include immunoglobulin molecules and immunologically active fragments of immunoglobulin molecules, i.e., molecules that contain an antigen-binding site. Immunoglobulin molecules can be of any type (e.g., IgG, IgE, IgM, IgD, IgA and IgY), class (e.g., IgG1, IgG2, IgG3, IgG4, IgM1, IgM2, IgA1 and IgA2) or subclass.

The term “host cell” means any cell that contains a heterologous nucleic acid. The heterologous nucleic acid can be a vector (e.g., an expression vector). For example, a host cell can be a cell from any organism that is selected, modified, transformed, grown, used or manipulated in any way, for the production of a substance by the cell, for example the expression by the cell of a gene, a DNA or RNA sequence, a protein or an enzyme. An appropriate host may be determined. For example, the host cell may be selected based on the vector backbone and the desired result. By way of example, a plasmid or cosmid can be introduced into a prokaryote host cell for replication of several types of vectors. Bacterial cells such as, but not limited to DH5α, JM109, and KCB, SURE® Competent Cells, and SOLOPACK Gold Cells, can be used as host cells for vector replication and/or expression. Additionally, bacterial cells such as E. coli LE392 could be used as host cells for phage viruses. Eukaryotic cells that can be used as host cells include, but are not limited to yeast (e.g., YPH499, YPH500 and YPH501), insects and mammals. Examples of mammalian eukaryotic host cells for replication and/or expression of a vector include, but are not limited to, HeLa, NIH3T3, Jurkat, 293, COS, CHO, Saos, and PC12.

The terms “express” and “expression” mean allowing or causing the information in a gene or DNA sequence to become produced, for example producing a protein by activating the cellular functions involved in transcription and translation of a corresponding gene or DNA sequence. A DNA sequence is expressed in or by a cell to form an “expression product” such as a protein. The expression product itself, e.g., the resulting protein, may also be said to be “expressed” by the cell. An expression product can be characterized as intracellular, extracellular or transmembrane.

The term “transfection” means the introduction of a “foreign” (i.e., extrinsic or extracellular) nucleic acid into a cell using recombinant DNA technology. The term “genetic modification” means the introduction of a “foreign” (i.e., extrinsic or extracellular) gene, DNA or RNA sequence to a host cell, so that the host cell will express the introduced gene or sequence to produce a desired substance, typically a protein or enzyme coded by the introduced gene or sequence. The introduced gene or sequence may also be called a “cloned” or “foreign” gene or sequence, may include regulatory or control sequences operably linked to polynucleotide encoding the chimeric antigen receptor, such as start, stop, promoter, signal, secretion, or other sequences used by a cell's genetic machinery. The gene or sequence may include nonfunctional sequences or sequences with no known function. A host cell that receives and expresses introduced DNA or RNA has been “genetically engineered.” The DNA or RNA introduced to a host cell can come from any source, including cells of the same genus or species as the host cell, or from a different genus or species.

The term “transduction” means the introduction of a foreign nucleic acid into a cell using a viral vector.

The term “regulatory element” refers to any cis-acting genetic element that controls some aspect of the expression of nucleic acid sequences. In some embodiments, the term “promoter” comprises essentially the minimal sequences required to initiate transcription. In some embodiments, the term “promoter” includes the sequences to start transcription, and in addition, also include sequences that can upregulate or downregulate transcription, commonly termed “enhancer elements” and “repressor elements”, respectively.

As used herein, the term “operatively linked,” and similar phrases, when used in reference to nucleic acids or amino acids, refer to the operational linkage of nucleic acid sequences or amino acid sequence, respectively, placed in functional relationships with each other. For example, an operatively linked promoter, enhancer elements, open reading frame, 5′ and 3′ UTR, and terminator sequences result in the accurate production of a nucleic acid molecule (e.g., RNA). In some embodiments, operatively linked nucleic acid elements result in the transcription of an open reading frame and ultimately the production of a polypeptide (i.e., expression of the open reading frame). As another example, an operatively linked peptide is one in which the functional domains are placed with appropriate distance from each other to impart the intended function of each domain.

By “enhance” or “promote,” or “increase” or “expand” or “improve” refers generally to the ability of a composition contemplated herein to produce, elicit, or cause a greater physiological response (i.e., downstream effects) compared to the response caused by either vehicle or a control molecule/composition. A measurable physiological response may include an increase in T cell expansion, activation, effector function, persistence, and/or an increase in cancer cell death killing ability, among others apparent from the understanding in the art and the description herein. In certain embodiments, an “increased” or “enhanced” amount can be a “statistically significant” amount, and may include an increase that is 1.1, 1.2, 1.5, 2, 3, 4, 5, 6, 7, 8, 9, 10, 15, 20, 30, or more times (e.g., 500, 1000 times) (including all integers and decimal points in-between and above 1, e.g., 1.5, 1.6, 1.7. 1.8, etc.) the response produced by vehicle or a control composition.

By “decrease” or “lower,” or “lessen,” or “reduce,” or “abate” refers generally to the ability of composition contemplated herein to produce, elicit, or cause a lesser physiological response (i.e., downstream effects) compared to the response caused by either vehicle or a control molecule/composition. In certain embodiments, a “decrease” or “reduced” amount can be a “statistically significant” amount, and may include a decrease that is 1.1, 1.2, 1.5, 2, 3, 4, 5, 6, 7, 8, 9, 10, 15, 20, 30 or more times (e.g., 500, 1000 times) (including all integers and decimal points in-between and above 1, e.g., 1.5, 1.6, 1.7. 1.8, etc.) the response (reference response) produced by vehicle, a control composition, or the response in a particular cell lineage.

The term “effective” applied to dose or amount refers to that quantity of a compound or pharmaceutical composition that is sufficient to result in a desired activity upon administration to a subject in need thereof. Note that when a combination of active ingredients is administered, the effective amount of the combination may or may not include amounts of each ingredient that would have been effective if administered individually. The exact amount required will vary from subject to subject, depending on the species, age, and general condition of the subject, the severity of the condition being treated, the particular drug or drugs employed, the mode of administration, and the like.

The phrase “pharmaceutically acceptable”, as used in connection with compositions described herein, refers to molecular entities and other ingredients of such compositions that are physiologically tolerable and do not typically produce untoward reactions when administered to a mammal (e.g., a human). Preferably, the term “pharmaceutically acceptable” means approved by a regulatory agency of the Federal or a state government or listed in the U.S. Pharmacopeia or other generally recognized pharmacopeia for use in mammals, and more particularly in humans.

The term “protein” is used herein encompasses all kinds of naturally occurring and synthetic proteins, including protein fragments of all lengths, fusion proteins and modified proteins, including without limitation, glycoproteins, as well as all other types of modified proteins (e.g., proteins resulting from phosphorylation, acetylation, myristoylation, palmitoylation, glycosylation, oxidation, formylation, amidation, polyglutamylation, ADP-ribosylation, pegylation, biotinylation, etc.).

The terms “nucleic acid”, “nucleotide”, and “polynucleotide” encompass both DNA and RNA unless specified otherwise. By a “nucleic acid sequence” or “nucleotide sequence” is meant the nucleic acid sequence encoding an amino acid; these terms may also refer to the nucleic acid sequence including the portion coding for any amino acids added as an artifact of cloning, including any amino acids coded for by linkers.

The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Alternatively, the carrier can be a solid dosage form carrier, including but not limited to one or more of a binder (for compressed pills), a glidant, an encapsulating agent, a flavorant, and a colorant. Suitable pharmaceutical carriers are described in “Remington's Pharmaceutical Sciences” by E. W. Martin.

The term “about” or “approximately” includes being within a statistically meaningful range of a value. Such a range can be within an order of magnitude, preferably within 50%, more preferably within 20%, still more preferably within 10%, and even more preferably within 5% of a given value or range. The allowable variation encompassed by the term “about” or “approximately” depends on the particular system under study, and can be readily appreciated by one of ordinary skill in the art.

“Cluster of Differentiation 79B protein” or “CD79b” refers to a known protein which is also called B-Cell-Specific Glycoprotein B29, Ig-Beta, or AGM6. The B lymphocyte antigen receptor is a multimeric complex that includes the antigen-specific component, surface immunoglobulin (Ig), which non-covalently associates with Ig-alpha (Igα) and Ig-beta (Igβ). CD79b is the Igβ protein of the B-cell antigen component. All CD79b isoforms and variants are encompassed in “CD79b”. The amino acid sequences of the various isoforms are retrievable from GenBank accession numbers AAH32651.1, EAW94232.1, AAH02975.2, NP 000617.1, and NP_001035022.1 The amino acid sequence of a full length CD79b sequence is shown in SEQ ID NO: 33. The sequence includes the extracellular domain (residues 29-159) and the cytoplasmic domain (residues 181-229).

The term “CD9B441” refers to any antibody, antigen-binding portion thereof, or any other protein that contains variable regions derived from CD9B441 VL (SEQ ID NO: 29) and CD9B441 VH (SEQ ID NO: 30), including a CAR. In certain embodiments, an anti-idiotype antibody of the disclosure specifically binds a protein comprising a VL domain as set forth in SEQ ID NO: 29 and/or a VH domain as set forth in SEQ ID NO: 30. In certain embodiments, an anti-idiotype antibody of the disclosure specifically binds a protein comprising the 3 CDRs of the VL domain set forth in SEQ ID NO: 29 and the 3 CDRs of the VH domain set forth in SEQ ID NO: 30.

The term “CD9B503” refers to a CD9B441-derived scFv-fusion protein, specifically CD9B441-HL (SEQ ID NO: 32).

The term “A003B192” refers to chimeric monoclonal antibodies (mAbs) with human VH (SEQ ID NO: 23)/VL (SEQ ID NO: 8) targeting CD9B441-derived scFv CD9B503 and murine IgG2a/k.

The term “A003B274” refers to chimeric monoclonal antibodies (mAbs) with human VH (SEQ ID NO: 24)/VL (SEQ ID NO: 8) targeting CD9B441-derived scFv CD9B503 and murine IgG2a/k.

The terminology used herein is for the purpose of describing particular embodiments only and is not intended to be limiting. As used herein, the indefinite articles “a”, “an” and “the” should be understood to include plural reference unless the context clearly indicates otherwise.

The disclosure further provides variants, e.g., functional variants, of the antibodies, nucleic acids, polypeptides, and proteins described herein. “Variant” refers to a polypeptide or a polynucleotide that differs from a reference polypeptide or a reference polynucleotide by one or more modifications for example, substitutions, insertions or deletions. The term “functional variant” as used herein refers to an antibody polypeptide, or protein having substantial or significant sequence identity or similarity to a parent antibody, polypeptide, or protein, which functional variant retains the biological activity of the antibody, polypeptide, or protein for which it is a variant. Functional variants encompass, e.g., those variants of the antibody, polypeptide, or protein described herein (the parent antibody, polypeptide, or protein) that retain the ability to recognize target cells to a similar extent, the same extent, or to a higher extent, as the parent antibody, polypeptide, or protein. In reference to the parent antibody, polypeptide, or protein, the functional variant can, for example, be at least about 30%, about 40%, about 50%, about 60%, about 75%, about 80%, about 85%, about 90%, about 91%, about 92%, about 93%, about 94%, about 95%, about 96%, about 97%, about 98%, about 99% or more identical in amino acid sequence to the parent antibody, polypeptide, or protein.

Herein, the structure of polypeptides is in places defined on the basis of % sequence identity with a recited reference sequence (with a given SEQ ID NO). In this context, % sequence identity between two amino acid sequences may be determined by comparing these two sequences aligned in an optimum manner and in which the amino acid sequence to be compared can comprise additions or deletions with respect to the reference sequence for an optimum alignment between these two sequences. The percentage of identity is calculated by determining the number of identical positions for which the amino acid residue is identical between the two sequences, by dividing this number of identical positions by the total number of positions in the comparison window and by multiplying the result obtained by 100 in order to obtain the percentage of identity between these two sequences. Typically, the comparison window with correspond to the full length of the sequence being compared. For example, it is possible to use the BLAST program, “BLAST 2 sequences” (Tatusova et al, “Blast 2 sequences—a new tool for comparing protein and nucleotide sequences”, FEMS Microbiol Lett. 174:247-250) available on the site http://www.ncbi.nlm.nih.gov/gorf/b12.html, the parameters used being those given by default (in particular for the parameters “open gap penalty”: 5, and “extension gap penalty”: 2; the matrix chosen being, for example, the matrix “BLOSUM 62” proposed by the program), the percentage of identity between the two sequences to be compared being calculated directly by the program. Determining sequence identity of a query sequence to a reference sequence is within the ability of the skilled person and can be performed using commercially available analysis software such as BLAST™.

A functional variant can, for example, comprise the amino acid sequence of the parent antibody, polypeptide, or protein with at least one conservative amino acid substitution. In another embodiment, the functional variants can comprise the amino acid sequence of the parent antibody, polypeptide, or protein with at least one non-conservative amino acid substitution. In this case, the non-conservative amino acid substitution may not interfere with or inhibit the biological activity of the functional variant. The non-conservative amino acid substitution may enhance the biological activity of the functional variant such that the biological activity of the functional variant is increased as compared to the parent antibody, polypeptide, or protein.

Amino acid substitutions of the inventive antibodies may be conservative amino acid substitutions. Conservative amino acid substitutions are known in the art, and include amino acid substitutions in which one amino acid having certain physical and/or chemical properties is exchanged for another amino acid that has the same or similar chemical or physical properties. For example, the conservative amino acid substitution can be an acidic amino acid substituted for another acidic amino acid (e.g., Asp or Glu), an amino acid with a nonpolar side chain substituted for another amino acid with a nonpolar side chain (e.g., Ala, Gly, Val, Ile, Leu, Met, Phe, Pro, Trp, Val, etc.), a basic amino acid substituted for another basic amino acid (Lys, Arg, etc.), an amino acid with a polar side chain substituted for another amino acid with a polar side chain (Asn, Cys, Gln, Ser, Thr, Tyr, etc.), etc.

The antibodies, polypeptides, and proteins of embodiments of the invention (including functional portions and functional variants of the invention) can comprise synthetic amino acids in place of one or more naturally-occurring amino acids. Such synthetic amino acids are known in the art, and include, for example, aminocyclohexane carboxylic acid, norleucine, α-amino n-decanoic acid, homoserine, S-acetylaminomethyl-cysteine, trans-3- and trans-4-hydroxyproline, 4-aminophenylalanine, 4-nitrophenylalanine, α-(2-amino-2-norbornane)-carboxylic acid, α,γ-diaminobutyric acid, α,β-diaminopropionic acid, homophenylalanine, 4-chlorophenylalanine, 4-carboxyphenylalanine, β-phenyl serine β-hydroxyphenylalanine, phenylglycine, α-naphthylalanine, cyclohexylalanine, cyclohexylglycine, N′-benzyl-N′-methyl-lysine, N′,N′-dibenzyl-lysine, 6-hydroxylysine, ornithine, α-aminocyclopentane carboxylic acid, α-aminocyclohexane carboxylic acid, α-aminocycloheptane carboxylic acid, indoline-2-carboxylic acid, 1,2,3,4-tetrahydroisoquinoline-3-carboxylic acid, aminomalonic acid, aminomalonic acid monoamide, and α-tert-butylglycine.

The antibodies, polypeptides, and proteins of embodiments of the invention (including functional portions and functional variants) can be subject to post-translational modifications. They can be glycosylated, esterified, N-acylated, amidated, carboxylated, phosphorylated, esterified, cyclized via, e.g., a disulfide bridge, or converted into an acid addition salt. In some embodiments, they are dimerized or polymerized, or conjugated.

The antibodies, polypeptides, and/or proteins of embodiments of the invention (including functional portions and functional variants thereof) can be obtained by methods known in the art. Suitable methods of de novo synthesizing polypeptides and proteins are described in references, such as Chan et al., Fmoc Solid Phase Peptide Synthesis, Oxford University Press, Oxford, United Kingdom, 2000; Peptide and Protein Drug Analysis, ed. Reid, R., Marcel Dekker, Inc., 2000; and Epitope Mapping, ed. Westwood et al., Oxford University Press, Oxford, United Kingdom, 2001. Also, polypeptides and proteins can be recombinantly produced using the nucleic acids described herein using standard recombinant methods. See, for instance, Sambrook et al., Molecular Cloning: A Laboratory Manual, 3rd ed., Cold Spring Harbor Press, Cold Spring Harbor, N.Y. 2001; and Ausubel et al., Current Protocols in Molecular Biology, Greene Publishing Associates and John Wiley & Sons, N Y, 1994. Further, some of the antibodies, polypeptides, and proteins of the invention (including functional portions and functional variants thereof) can be isolated and/or purified from a source, such as a plant, a bacterium, an insect, a mammal, etc. Methods of isolation and purification are known in the art. Alternatively, the antibodies, polypeptides, and/or proteins described herein (including functional portions and functional variants thereof) can be commercially synthesized. In this respect, the antibodies, polypeptides, and proteins can be synthetic, recombinant, isolated, and/or purified.

### Methods and Uses of the Disclosure

The disclosure provides anti-idiotype antibodies and antigen-binding portions thereof that specifically bind a CD9B441 containing protein, e.g., an antibody or antigen-binding portions thereof. For example, the anti-idiotype antibodies may include amino acid sequences complementary to portions of a CD9B441 antibody to facilitate specific binding. The disclosure also provides nucleic acids encoding the anti-idiotype antibodies and antigen-binding portions thereof, methods of producing the anti-idiotype antibodies and antigen-binding portions thereof, methods of detecting CD9B441 using the anti-idiotype antibodies and antigen-binding portions thereof and kits including the anti-idiotype antibodies and antigen-binding portions thereof. For example, the anti-idiotype antibodies may be included in kits containing other reagents and used to determine whether a given biological sample includes CD9B441/CD9B503 antibodies or fragments thereof, for example, expressed on the surface of a T cell in a CAR.

Methods of testing antibodies for the ability to bind to any functional portion of CD9B441 are known in the art and include any antibody-antigen binding assay, such as, for example, radioimmunoassay (MA), Western blot, enzyme-linked immunosorbent assay (ELISA), immunoprecipitation, and competitive inhibition assays.

Suitable methods of making antibodies are known in the art. For instance, standard hybridoma methods are described in, e.g., Köhler and Milstein, Eur. J. Immunol., 5, 511-519 (1976), Harlow and Lane (eds.), Antibodies: A Laboratory Manual, CSH Press (1988), and C. A. Janeway et al. (eds.), Immunobiology, 5th Ed., Garland Publishing, New York, N.Y. (2001)). Alternatively, other methods, such as EBV-hybridoma methods (Haskard and Archer, J. Immunol. Methods, 74(2), 361-67 (1984), and Roder et al., Methods Enzymol., 121, 140-67 (1986)), and bacteriophage vector expression systems (see, e.g., Huse et al., Science, 246, 1275-81 (1989)) are known in the art. Further, methods of producing antibodies in non-human animals are described in, e.g., U.S. Pat. Nos. 5,545,806, 5,569,825, and 5,714,352, and U.S. Patent Application Publication No. 2002/0197266 A1).

Phage display can also be used to generate an antibody. In this regard, phage libraries encoding antigen-binding variable (V) domains of antibodies can be generated using standard molecular biology and recombinant DNA techniques (see, e.g., Sambrook et al., supra, and Ausubel et al., supra). Phage encoding a variable region with the desired specificity are selected for specific binding to the desired antigen, and a complete or partial antibody is reconstituted comprising the selected variable domain. Nucleic acid sequences encoding the reconstituted antibody are introduced into a suitable cell line, such as a myeloma cell used for hybridoma production, such that antibodies having the characteristics of monoclonal antibodies are secreted by the cell (see, e.g., Janeway et al., supra, Huse et al., supra, and U.S. Pat. No. 6,265,150).

In one aspect, the disclosure provides an anti-idiotype antibody or antigen-binding portion thereof that specifically binds a target antibody or CAR that comprises CD9B441. For example, the anti-idiotype antibody or antigen-binding portion may specifically bind one or more of the domains of the fragment antigen-binding region (Fab), including the VH and VL. In some embodiments, the anti-idiotype antibody or antigen-binding portion comprises a VL domain with the amino acid sequence of SEQ ID NO: 8 and a VH domain with an amino acid sequence selected from the group consisting of SEQ ID NOs: 23-24.

In other embodiments, the anti-idiotype antibody or antigen-binding portion is for use in detecting CD9B441 in a biologic sample comprising: (a) providing a biological sample; (b) contacting the biological sample with the anti-idiotype antibody or antigen-binding portion; and (c) detecting the anti-idiotype antibody or antigen-binding portion. For example, an anti-idiotype antibody may be added to any biologic sample, including: a tissue sample, a tumor sample, a cell or a fluid with other biological components, organisms, subunits of proteins/antigens, killed or inactivated whole cells or lysates. The anti-idiotype antibody may be contained in a solution containing pharmaceutically acceptable reagents, including but not limited to buffers, a stabilizer, and/or polymers. The anti-idiotype antibody may be contacted by pipetting and/or mixing with the biologic sample. Then anti-idiotype antibody may then specifically bind a CD9B441 containing protein, e.g., an antibody or antigen-binding portions thereof, in the biologic sample. As one example, whether an anti-idiotype antibody has bound to CD9B441 may be determined by washing unbound anti-idiotype antibody, leaving only complexed anti-idiotype antibody. Continuing with this example, the anti-idiotype antibody may include a fluorophore, which may be illuminated to give a signal proportional to the amount of CD9B441 in the biologic sample. Detection of a bound complex of anti-idiotype antibody to CD9B441 is described further below.

In another aspect, the disclosure provides an anti-idiotype antibody or antigen-binding portion thereof that specifically binds to an anti-CD79b antibody that comprises a light chain variable (VL) domain comprising the LCDR1 of SEQ ID NO: 5, the LCDR2 of SEQ ID NO: 6, and the LCDR3 of SEQ ID NO: 7; and further comprises a heavy chain variable (VH) domain comprising an HCDR1 having an amino acid sequence selected from the group consisting of SEQ ID NOs: 17-18, an HCDR2 having an amino acid sequence selected from the group consisting of SEQ ID NOs: 19-20; and an HCDR3 having an amino acid sequence selected from the group consisting of SEQ ID NOs: 21-22.

In some embodiments, the disclosure provides an anti-idiotype antibody or antigen-binding portion thereof that specifically binds to an anti-CD79b antibody, wherein the anti-idiotype antibody or antigen-binding portion comprises a complementarity determining region of a light chain variable (VL) domain comprising, the LCDR1 of SEQ ID NO: 5, the LCDR2 of SEQ ID NO: 6, the LCDR3 of SEQ ID NO: 7; and further comprises a complementarity determining region of a heavy chain variable (VH) domain comprising HCDR1-3 having amino acid sequences selected from the group consisting of an HCDR1 having the amino acid sequence of SEQ ID NO: 17, an HCDR2 having the amino acid sequence of SEQ ID NO: 19, an HCDR3 having the amino acid sequence of SEQ ID NO: 21; and an HCDR1 having the amino acid sequence of SEQ ID NO: 18, an HCDR2 having the amino acid sequence of SEQ ID NO: 20, and an HCDR3 having the amino acid sequence of SEQ ID NO: 22.

In certain embodiments, the disclosure provides an anti-idiotype antibody or antigen-binding portion thereof that specifically binds to an anti-CD79b antibody that comprises a light chain variable (VL) domain comprising the LCDR1 of SEQ ID NO: 5, the LCDR2 of SEQ ID NO: 6, the LCDR3 of SEQ ID NO: 7, and further comprises a heavy chain variable (VH) domain comprising the HCDR1 of SEQ ID NO: 17, the HCDR2 of SEQ ID NO: 19, and the HCDR3 of SEQ ID NO: 21.

In certain embodiments, the disclosure provides an anti-idiotype antibody or antigen-binding portion thereof that specifically binds to an anti-CD79b antibody that comprises a light chain variable (VL) domain comprising the LCDR1 of SEQ ID NO: 5, the LCDR2 of SEQ ID NO: 6, the LCDR3 of SEQ ID NO: 7, and further comprises a heavy chain variable (VH) domain comprising the HCDR1 of SEQ ID NO: 18, the HCDR2 of SEQ ID NO: 20, and the HCDR3 of SEQ ID NO: 22.

In some embodiments, the disclosure provides an anti-idiotype antibody or antigen-binding portion thereof that specifically binds to an anti-CD79b antibody that comprises the light chain variable (VL) domain of SEQ ID NO: 8; and further comprises a heavy chain variable (VH) domain comprising the HFW1 of SEQ ID NO: 11, an HFW2 having an amino acid sequence selected from the group consisting of SEQ IQ NOs: 12-13, an HFW3 having the amino acid sequence selected from the group consisting of SEQ ID NOs: 14-15, the HFW4 of SEQ ID NO: 16, an HCDR1 having an amino acid sequence selected from the group consisting of SEQ ID NOs: 17-18, an HCDR2 having an amino acid sequence selected from the group consisting of SEQ ID NOs: 19-20, an HCDR3 having the amino acid sequence selected from the group consisting of SEQ ID NOs: 21-22.

In some embodiments, the anti-idiotype antibody or antigen-binding portion thereof that specifically binds to an anti-CD79b antibody comprises a VL domain that has an amino acid sequence having at least 90% sequence identity to SEQ ID NO: 8 and a VH domain that has an amino acid sequence having at least 90% sequence identity to SEQ ID NO: 23. In some embodiments, the anti-idiotype antibody or antigen-binding portion thereof that specifically binds to an anti-CD79b antibody comprises a light chain that has an amino acid sequence having at least 90% sequence identity to SEQ ID NO: 9 and a heavy chain that has an amino acid sequence having at least 90% identity to SEQ ID NO: 25.

In certain embodiments, the anti-idiotype antibody or antigen-binding portion thereof that specifically binds to an anti-CD79b antibody comprises the VL domain of SEQ ID NO: 8 and the VH domain of SEQ ID NO: 23. In certain embodiments, the anti-idiotype antibody or antigen-binding portion thereof that specifically binds to an anti-CD79b antibody comprises the light chain of SEQ ID NO: 9 and the heavy chain of SEQ ID NO: 25.

In some embodiments, the anti-idiotype antibody or antigen-binding portion thereof that specifically binds to an anti-CD79b antibody comprises a VL domain that has an amino acid sequence having at least 90% sequence identity to SEQ ID NO: 8 and a VH domain that has an amino acid sequence having at least 90% sequence identity to SEQ ID NO: 24. In some embodiments, the anti-idiotype antibody or antigen-binding portion thereof that specifically binds to an anti-CD79b antibody comprises a light chain that has an amino acid sequence having at least 90% sequence identity to SEQ ID NO: 9 and a heavy chain that has an amino acid sequence having at least 90% identity to SEQ ID NO: 26.

In certain embodiments, the anti-idiotype antibody or antigen-binding portion thereof that specifically binds to an anti-CD79b antibody comprises the VL domain of SEQ ID NO: 8 and the VH domain of SEQ ID NO: 24. In certain embodiments, the anti-idiotype antibody or antigen-binding portion thereof that specifically binds to an anti-CD79b antibody comprises the light chain of SEQ ID NO: 9 and the heavy chain of SEQ ID NO: 26.

In some embodiments, the antigen-binding portion is selected from a Fab, F(ab′)₂, or scFv. In some embodiments, the antibody is a monoclonal antibody. In some embodiments, the antibody is a chimeric antibody. In some embodiments, the chimeric antibody comprises a murine IgG2a framework. In certain embodiments, the murine IgG2a framework may include a murine Ig heavy chain signal peptide from mix FVB/N, C57BL/6J comprising the sequence MAWVWTLLFLMAAAQSIQA.

In some other embodiments, the antibody is a fully human antibody. For example, the fully human antibody may be an IgG, IgM, IgA, IgE, or IgD. In some embodiments, the anti-idiotype antibody or antigen-binding portion thereof is specific to CD9B441, wherein CD9B441 is within the antigen-binding domain of the extracellular portion of a chimeric antigen receptor (CAR). For example, a CD9B441 encoding nucleic acid may be introduced in vitro into a T-cell, of which at least some portion then is expressed on the extracellular portion of a CAR. The anti-idiotype antibody may then specifically bind the extracellular portion of the CAR. In some embodiments, CD9B441 is an scFv and the anti-idiotype antibody or antigen-binding portion specifically binds an epitope in the scFv of the CAR. In some embodiments, CD9B441 specifically binds CD79b. In some embodiments, the anti-idiotype antibody or antigen-binding portion does not cross-react to other CD79b antibodies or other CD79b binding CARs. For example, to prevent false positives in an assay for determining whether CD9B441 has been expressed on the extracellular portion of a CAR, the anti-idiotype antibody may be specific to CD9B441, and not have appreciable binding to its target CD79b or other CD79b targeting ligands that are not CD9B441. In some embodiments, the CAR has an amino acid sequence selected from the group consisting of SEQ ID NOs: 31-32.

In some embodiments, the disclosure provides a nucleic acid encoding the heavy chain, the light chain, or both, of the anti-idiotype antibody or antigen-binding portion. For example, the nucleic acid may be DNA, RNA, and any chemical modifications thereto (e.g., nucleoside modifications).

In another aspect, the disclosure provides a nucleic acid encoding the heavy chain, the light chain, or both, of an anti-idiotype antibody or an antigen-binding portion thereof that specifically binds CD9B441, wherein said nucleic acid comprises: the nucleotide sequence of SEQ ID NO: 10; the nucleotide sequence of SEQ ID NO: 27; or both. In some embodiments, the disclosure provides a nucleic acid encoding the heavy chain, the light chain, or both, of an anti-idiotype antibody or an antigen-binding portion thereof that specifically binds CD9B441, wherein said nucleic acid comprises: the nucleotide sequence of SEQ ID NO: 10; the nucleotide sequence of SEQ ID NO: 28; or both. In another aspect, the disclosure provides a vector comprising the nucleic acid sequence. For example, the vector may be a self-replicating nucleic acid structure, or incorporated into the genome of a host cell into which it has been introduced. In some embodiments, the vector is an expression vector. In another aspect, the disclosure provides a host cell comprising the vector. In some embodiments, the host cell is a mammalian cell.

In another aspect, the disclosure provides a method of producing an anti-idiotype antibody or antigen-binding portion thereof that specifically binds CD9B441, said method comprising culturing a host cell under conditions that allow said antibody or antigen-binding portion to be expressed, wherein the host cell comprises nucleotide sequences coding the heavy chain and light chain of the antibody or antigen-binding portion, and isolating said antibody or antigen-binding portion from the culture. For example, the anti-idiotype antibody may be produced by homogenous suspension culture in deep-tank stirred fermenters, perfusion-tank systems, airlift reactors, and continuous-culture systems. Anti-idiotype antibodies may be isolated from reaction and/or growth mixtures by physical or chemical separation procedures, including affinity separation using Protein A or G, size exclusion chromatography, and charge separations. In some embodiments, the host cell encodes a vector comprising a nucleic acid encoding the anti-idiotype antibody or antigen-binding portion thereof.

In another aspect, the disclosure provides a method for detecting CD9B441 in a biologic sample comprising: (a) providing a biological sample; (b) contacting the biological sample with the anti-idiotype antibody or antigen-binding portion; and (c) detecting the anti-idiotype antibody or antigen-binding portion. For example, the anti-idiotype antibody may bind CD9B441 expressed in the biological sample. The bound complex may be detected by any detection method, including both chemical and physical detection methods. For example, the detecting method may be used to identify the mere presence of the antibody of interest in a biological sample, or may be used to test whether the antibody of interest in a sample is present at a detectable level, or may be used to quantify the amount of the antibody of interest in a sample and further to compare the antibody levels from different samples. For example, the detecting method may be one or more of: immunoprecipitation, immunocytochemistry, immunoblotting, and immunosorbent assays. As a specific example, the immunsorbent assay may be an ELISA or ELISA-type assay that includes a bound anti-idiotype antibody or fragment thereof over which the biologic sample is washed.

In another aspect, the disclosure provides a method for detecting expression of a chimeric antigen receptor (CAR) comprising CD9B441 in a biologic sample comprising: (a) providing a biological sample; (b) contacting the biological sample with the anti-idiotype antibody or antigen-binding portion; and (c) detecting the anti-idiotype antibody or antigen-binding portion, and thereby detecting the expression of the CAR.

In some embodiments, the antibody comprises a detectable label. In some embodiments, the method further comprises further comprises contacting the anti-idiotype antibody or antigen-binding portion with a detectable label before detecting the anti-idiotype antibody or antigen-binding portion. For example, the detectable label may be any chemical tag or component that is either integral to, binds to, or otherwise complexes to the anti-idiotype antibody and emits or otherwise provides a unique identifiable signal. For example, the detectable label may be an isotope marker, colorimetric biosensor, photochromic compound, fluorescent label, fluorogenic label, or electrochemical sensor. As a specific example, the fluorescent label may be green fluorescent protein, yellow fluorescent protein, blue fluorescent protein, fluorescein, rhodamine, coumarin, cyanine, phycoerythrin, and derivatives thereof.

In some embodiments, the biological sample is blood, serum or urine. For example, the biological sample may be whole blood, serum, plasma, urine, feces, cerebrospinal fluid, ascites, and the like. In some embodiments, the biological sample is fresh biological material, such as biological material taken at a given time for the purpose of this analysis. The biological sample may also be biological material taken at another point during patient care for this or for other purposes, or using archived patient material. The biological sample may be freshly obtained or previously obtained, and where previously obtained may have been stored prior to use (e.g., at room temperature, refrigerated, or frozen).

In some aspects, the disclosure provides a kit for detecting CD9B441 in a biologic sample comprising: (a) an anti-idiotype antibody or antigen-binding portion; and (b) instructions for detecting the anti-idiotype antibody or antigen-binding portion. For example, the kit may include the anti-idiotype antibody or antigen-binding portion thereof as a solid powder, lyophilized powder, liquid solution, or liquid components to mix to form a solution, or bound to a solid support. The kit may include additional reagents, including stabilizers, buffers, and other pharmaceutically acceptable excipients needed to facilitate using the kit in an assay of a biological sample. The kit may also include written instructions directing a user on how to perform the assay.

In other aspects, the disclosure provides a method of purifying CD9B441 from a sample comprising: (a) providing a biological sample comprising CD9B441; (b) contacting the biological sample with an anti-idiotype antibody or antigen-binding portion of the disclosure; and (c) capturing the anti-idiotype antibody or antigen-binding portion, including a CAR or other protein that contains CD9B441, and thereby purifying CD9B441. For example, any separation method, including physical and chemical methods, may be used to capture the anti-idiotype antibody. Specifically, the CD9B441 can be captured and isolated from cell culture medium, host cells, or both using techniques known in the art for purifying proteins, including ion-exchange chromatography, gel filtration chromatography, ultrafiltration, electrophoresis, and immunoaffinity purification with antibodies specific for particular epitopes of the anti-idiotype antibody. In some embodiments, the anti-idiotype antibody is a fusion protein containing a domain which facilitates its purification. In certain embodiments, a purified CD9B441 composition is substantially isolated from proteins that do not contain CD9B441. In some embodiments, a purified CD9B441 composition is 100% pure, 99% pure, 98% pure, 97% pure, 96% pure, 95% pure, or 90% pure or greater.

In other aspects, the disclosure provides a method of selecting CAR-T cells from a cell population comprising: (a) providing a biological sample comprising CAR-T cells; (b) contacting the biological sample with an anti-idiotype antibody or antigen-binding portion; and (c) capturing the anti-idiotype antibody or antigen-binding portion, and thereby selecting CAR-T cells. In some embodiments, the anti-idiotype antibody or antigen-binding portion thereof is specific to CD9B441.

### EXAMPLES

### Example 1: Determination of CD9B441-Binding Fabs

Anti-CD9B441-derived scFv-fusion protein binding Fabs were selected from two sets of de novo Fab-pIX phage display libraries as described in Shi et al., J. Mol. Biol. 397:385-96, 2010; Int. Pat. Publ. No. WO2009/085462; U.S. Pat. Publ. No. US2010/0021477). CD9B441-HL is the lead molecule from wave 2 in the CD79b CAR-T program, and its scFv-fusion protein CD9B503 is the antigen used in the de novo Fab-pIX phage display library panning.

In the phage selections using purified recombinant antigens, biotinylated CD9B503 were used as “bait” to capture and immobilize the phage binders. After several selection rounds, a polyclonal phage ELISA using purified antigens was performed to detect the specific enrichment of individual panning library. The phage collected from those panning libraries which demonstrated enrichment for binders to CD9B503 were expressed in E. coli as soluble Fabs for primary screening. The monoclonal Fab lysates prepared from the enriched Fab libraries were screened in Meso Scale Discovery (MSD) based electro-chemiluminescent immunoassays (ECLIA) for binding to CD9B503 (CD9B441-HL-scFv Fc) but not to a similar negative control scFv fusion protein CD9B504 (CD9B449-HL-scFv Fc).

Phage Panning

Six individual panning experiments using six individual V3.0 and V5.0 de novo Fab phage libraries were panned against biotinylated CD9B503 according to standard protocol (Cheadle, E. J. et al. Antibody Engineering. 907, 645-666 (2012). Briefly, the phage libraries and paramagnetic streptavidin (SA) beads were blocked in 1% bovine serum albumin/50% Chemiblocker (Millipore cat #2170)/49% 1×TBST (Teknova cat #T0310) for one hour. The biotinylated target protein, CD9B503, was pre-captured on the blocked beads starting at 100 nM concentration, and diminishing the concentrations in subsequent panning rounds. At the same time, 100 nM each of biotinylated CD9B492, CD9B500 and CD9B504 were mixed and pre-captured on the blocked beads for used as negative selection. Each of the de novo phage libraries were pre-adsorbed to against the negative selection SA beads containing biotinylated CD9B492, CD9B500 and CD9B504. The negative selection SA-beads were discarded along with phage clones that bind to the beads, and the pre-adsorbed phage library was added to the target biotinylated CD9B503 SA beads with the presence of 1 uM of nonbiotinylated CD9B492, CD9B500 and CD9B504. After some incubation, the SA beads/CD9B503/phage library complex were washed in 1×TBST several times, depending on the panning round. After the final wash, bead-bound phage clones were rescued by infection of log phase TG1 E. coli cells (OD600 nm=0.4-0.6). The phage-infected TG1 cells were inoculated into 10 mls of 2×YT media with 100 μg/ml carbenicillin and 1% glucose then grown overnight at 37° C. with shaking. Phage were produced and subjected to additional panning. To increase selection pressure, the antigen concentration was reduced from 100 nM to 10 nM in each subsequent round (R #): R1 100 nM at 4° C. overnight; R2 10 nM at room temperature (RT) for 1 hour; R3 10 nM at 4° C. overnight.

Polyclonal Phage ELISA

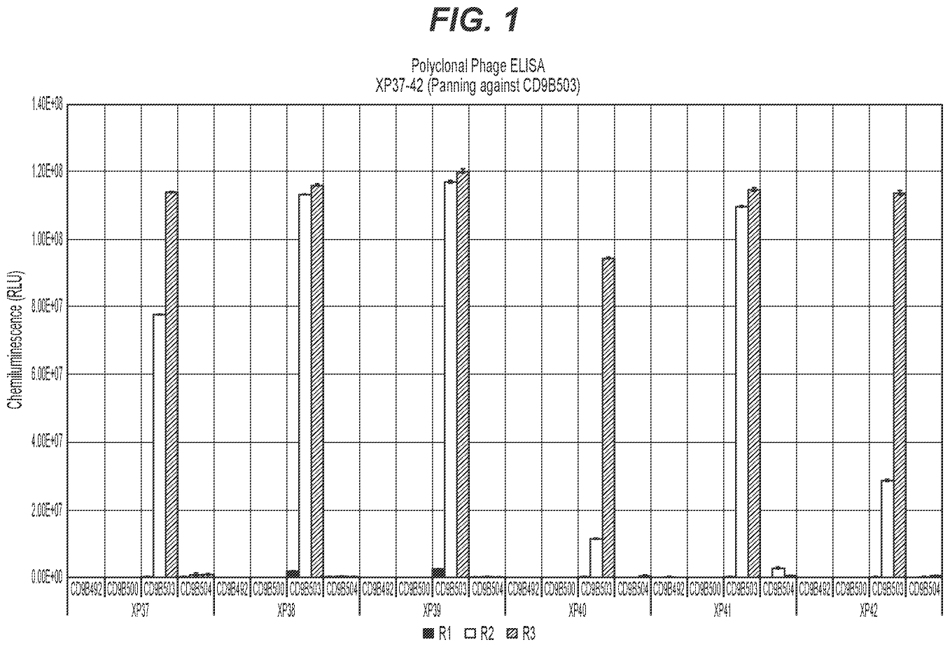

Enrichment of binders was determined from each panning experiment by polyclonal phage ELISA. Briefly, 100 μl of 20 nM biotinylated CD9B503 and counter screening reagents diluted in 1×TBS (Teknova cat #T9530) were captured on black NA-coated plate (Thermo cat #15217). After an hour incubation at 37° C., the plate was washed six times in 300 μl 1×TBST. 300 μl of blocking buffer (10% BSA solution/50% Chemiblocker/50% 1×TBST) was added to each well of the plate and incubated at room temperature for 1 hour. After blocking, 1004, of polyclonal phage output from each panning round diluted 1/100 in blocking buffer, were added to each well of the plate and incubated at room temperature for 1 hour to allow the binding of Fab displayed on the phage particles to the immobilized CD9B503. Following the incubation, the plate was washed 10 times with 1×TBST. 100 μl of HRP-conjugated anti-M13 (pVIII) antibody (GE Healthcare cat #27942101) diluted 1:2500 in blocking buffer was added to the plate and incubated at room temperature. After 1 hour incubation, the plate was washed 10 times with 300 μl of 1×TBST. 1004, of prepared BM chemiluminescence ELISA Substrate (Roche cat #11582950001) was added to each well of the plate. The chemiluminescence or relative light unit (RLU) was measured by Envision plate reader. As shown in FIG. 1, all six panning experiments showed CD9B503 specific binding enrichment after round 3 panning. Both A003B192 and A003B274 came from XP40 panning experiments.

Fab Production

Plasmid DNA was isolated and purified from glycerol stocks of specific rounds of phage panning experiments that were identified to demonstrate enrichment of binders to CD9B503, and transformed into TG-1 E. coli cells then grown on LB/Agar plates containing 100 ug/ml carbenicillin at 37° C. overnight. The overnight cultures were used for (i) colony PCR and sequencing of the V-regions, and (ii) starting culture for Fab production. For Fab production, the overnight culture was diluted 10 fold in fresh 2×YT media containing 100 ug/ml carbenicillin and grown for 5-6 hours at 37° C. Fab production was induced by the addition of fresh media containing 2 mM IPTG and 200 ug/ml carbenicillin, and the cultures were grown overnight at 30° C. The cultures were spun down and the bacterial pellet was lysed using BugBuster™ (Millipore) to release the soluble Fab proteins. The cell lysate was spun down and the supernatant were used for Fab ELISA.

Primary Screening

The monoclonal Fab lysates prepared from the enriched Fab libraries were screened in Meso Scale Discovery (MSD) based electro-chemiluminescent immunoassays (ECLIA) for binding to CD9B503 but not to a similar negative control scFv fusion protein CD9B504 (CD9B449-HL-scFv Fc). Biotinylated ScFv-Fc fusion antigens, target antigen CD9B503 or negative control scFv fusion protein counter screening reagent CD9B504, were diluted to a concentration of 2.5 nM in SuperBlock T20 (TBS) Blocking Buffer (Thermo cat #37536) and 50 ul/well was added to their respective MSD 384-well streptavidin plates (Meso Scale Discovery cat #L21SA-5) and incubated on a shaker for 30 min at RT. The plates were washed 1×80 ul/well 1×PBST and crude Fab lysates from E. coli expressions in 96-well plates were stamped into the 384-well assay plates in duplicate and incubated on a shaker for 1 hour at RT. The assay plates were washed 1×80 ul/well 1×PBST and 6 nM of SULFO-TAG Anti-human/NHP Kappa Antibody (Meso Scale cat #D20TF-6) added at 10 ul/well, and the plates incubated on a shaker for 1 hour at RT. The plates were washed 2×80 ul/well 1×PBST and 35 ul/well of 1×MSD Read Buffer T (Meso Scale cat #R92TC-1) added to each well and the plates analyzed on an MSD Sector 5600 plate reader. All liquid handling was performed on an Agilent Bravo system and washing of 384-well plates were handled on a BioTek 405 Select plate washer. Clones having signal greater than the average background signal plus three times standard deviation in the CD9B503 binding assay and signal less than the average background signal plus three times standard deviation of in the CD9B504 binding assay were selected for sequencing. Also, clones having binding signal ratio greater than ten of CD9B503 over CD9B504 were selected for sequencing.

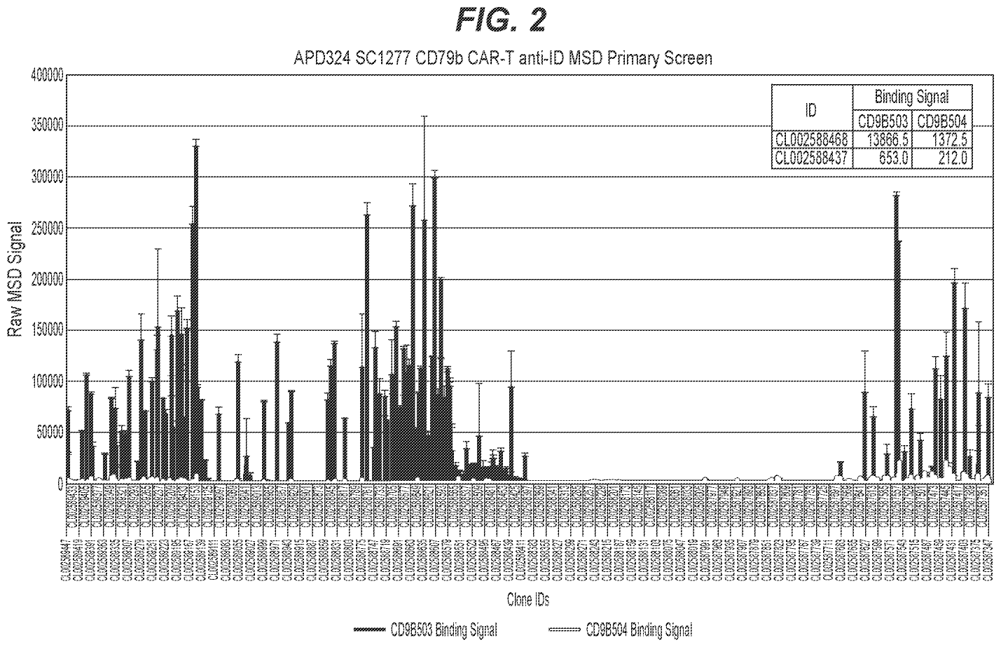

FIG. 2 shows the results of the results of the primary screen. Monoclonal Fabs were screened in MSD plates in binding to CD9B503 or CD9B504 (counter-screening). The average raw binding signals for the CL002588468 parent clone for the VH and VL in A003B192 and the CL002588437 parent clone for the VH and VL in A003B274 are shown in the embedded table, and both clones are from the ratio hit list (signal ratio greater than ten of CD9B503 over CD9B504).

### Example 2: Generation of Monoclonal Antibodies against CD9B503 (CD9B449-HL-scFv)

Fab Selection

The Fab clones that demonstrated binding activity to CD9B503 but not to CD9B504 were selected as hits. The selected Fabs were sequenced to determine V region sequences and identify unique clones. The unique Fab V regions were cloned into mammalian expression vectors to express as chimeric mAbs with murine IgG2a/murine Kappa constant regions.

The variable regions of A003B192 and A003B274 were identified through phage display using human Fab-pIX de novo libraries to soluble scFv-Fc fusion protein CD9B503. These V-regions did not undergo any affinity maturation. The DNA sequences were obtained from the de novo Fab library without any codon optimization.

Cloning of V_{H} and V_{L}

Two pcDNA3.1-derived mammalian expression vector (vDR000368 and vDR000961) were used to generate the single gene constructs encoding the heavy chain (HC) or light chain (LC) of the chimeric mAb. Each vector contains a human cytomegalovirus (hCMV) promoter to drive the expression of the HC and LC and both contain the ampicillin resistance gene (Amp(R)) to facilitate cloning. vDR000368 has unique HindIII and DraIII restriction enzyme sites for cloning a variable heavy (VH) chain into a mouse IgG2a constant region; vDR000961 has unique HindIII and Tth111I restriction enzyme sites for cloning variable light (VL) chain with a mouse Kappa constant region.

DNA fragments comprising the VH or VL of A003B192 and A003B274 were synthesized by IDT and ligated into HC vector vDR000368 and LC vector vDR000961. The HC synthetic fragment included a HindIII restriction enzyme site, Kozak sequence, DNA sequences encoding a signal peptide, the VH, part of the CH1, and a DraIII cloning site. The LC synthetic fragment included a HindIII restriction enzyme site, Kozak sequence, DNA sequences encoding a signal peptide, the VL, part of the Kappa constant region, and a Tth111I restriction cloning site. For A003B192, the final HC construct is PBD000109791, and the LC construct is PBD00098715. For A003B274, the final HC construct is PBD000109922, and the LC construct is PBD00098715. The HC and LC constructs were co-transfected in mammalian expression cell lines HEK293 Expi or CHO to make the final mAbs.

Protein Expression

The HC and LC constructs were sequenced verified before transfection. HEK Expi293™ cells (Thermo cat #A14527) grown in Expi293™ Expression media (Thermo cat #A1435101). The cells are grown at 37° C. shaking at 125 RPM with 8% CO₂. The cells were transfected at 2.5×10⁶ cells per ml using Expi293™ Expression Kit (Thermo cat #A14524). For each liter of cells transfected, 1 mg of total DNA was diluted in 25 ml of Opti-MEM media (Thermo cat #319850620) and 2.6 ml of Expi293™ reagent was diluted in 25 ml of Opti-MEM and incubated for 5 minutes at room temperature. The diluted DNA and diluted Expi293 reagent were combined and incubated for 20 minutes at room temperature. The DNA complex was then added to the cell. The cells were placed in the shaking incubator overnight. The next day after transfection, 5 ml of Enhancer 1 was diluted into 50 ml of Enhancer 2 and the total volume of the two Enhancers were added to the cells (Enhancers from Expression kit, Thermo cat #A14524). The transfected cells were placed back into the incubator for 4 days until harvested. The cells were removed by centrifugation at 4,500 g for 35 minutes then filtered with a 0.2 μm filter prior to checking expression levels.

For screening purposes, sometimes clones were transfected into 96-well deep well plates first. The same cells and reagents were used as described above. The plates were shaken at 1000 RPM. The plates were clarified by centrifugation for 30 minutes at 4,000 g, then filtered through a 0.2 μm filter plate.

Expression was quantitated by Octet. Murine IgG2 (Sigma cat #M9144) was used as the standard. Protein A biosensors were used. The samples and the standard were diluted with spent Expi293 media. The standard curve started at 100 ug/ml in a twofold dilution. The samples were diluted 1:10. The standard curve was a linear point curve. The calculations were performed by Forte Biosystems software.

### Example 3: Binding Assays for Anti-Idiotypic Antibodies A003B192 and A003B274

Binding Assay Using Soluble Proteins with Biacore

Biacore system (Cytiva) was used to measure bio-molecular interactions by surface plasmon resonance (SPR). Anti-Mouse Fc antibody directly immobilized on a CMS sensor chip. Library Antibodies (Mouse IgG2a) diluted to 1 ug/ml to reach approximately 20-60 RU. Antigens (scFv Fc fusions) are associated at 100 nM to 0.16 nM, 1:5 dilutions for 3 minutes. Dissociation for 30 mins. The results of the assay (Table 1 below) show the nanomolar dissociation constant of A003B192 and A003B274 with CD9B503 (CD9B441-HL-scFv-Fc bivalent fusion protein) and CD9B574 (CD9B441-LH-scFv-Fc monovalent fusion protein).

TABLE 1
Biacore binding results for anti-idiotypic antibodies A003B192
and A003B274 to CD9B441-scFv-Fc fusion proteins

| | SPR on CD9B503 | | SPR on CD9B574 | |
|---|---|---|---|---|
| Protein AA | (Bivalent) | | (Monovalent) | |
| Name | KD (nM) | KD (pM) | KD (nM) | KD (pM) |
| A003B192 | 0.04 | 37 | 1.6 | 1607 |
| A003B274 | 0.23 | 226 | NA | NA |

Cell Binding Assay

ScFv transfected SupT1-CD9B441-HL and SupT1-CD9B337-HL cells were cultured in RMPI 1640 (ATCC), 10% FBS, 1% Non-Essential Amino Acids, 1 mM Sodium Pyruvate, 2 mM L-glutamine, 10 mM HEPES, 0.1% bicarbonate. Cell culture supplements were ThermoFisher Scientific Gibco products. The mAbs were diluted to 6 micrograms/mL in Stain Buffer (BSA) (BD Pharmingen cat #554657). ScFv expressing SupT1 cells were labeled with fixable Live/Dead stain (Molecular Probes cat #L34974) and added to a 384-well V-bottom microplate (Greiner Bio-One cat #781281) at 50,000 cells/well.

The normalized mAb samples were added in 20 ul/well volume to the cell suspensions with gentle mixing and the cells incubated on ice for 30 min with a final mAb concentration 2.5 ug/ml. The cell suspension was diluted with 70 ul/well ice cold Stain Buffer (BSA), cells pelleted at 400×g for 5 min at 4° C. and the supernatant aspirated. The cell pellets were washed once more with 70 ul/well ice cold Stain Buffer (BSA). 3 ug/ml of AF488 anti-mouse IgG (H+L) specific goat F(ab′)₂ (Jackson ImmunoResearch cat #115-546-062) was added to the cell pellets at 40 ul/well with gentle mixing and the cells incubated on ice, in the dark, for 30 min. The cells were washed as already described and fixed with 40 ul/well BD Cytofix (BD Pharmingen cat #554655) for 20 min on ice. The fixed cells were washed as described above and the cell pellets resuspended in 20 ul Stain Buffer and analyzed on an iQue PLUS VBR flow cytometer (Sartorius). Data were analyzed using iQue Forecyte® 7.1 software for mean fluorescence intensities (MFI) for antibody binding in the BL1-H (AF488) channel gated on live and singlet populations. All liquid handling was performed on an Agilent Bravo system and aspiration of 384-well plates were handled on a BioTek 405 Select plate washer.

As shown in Table 2, A003B192 and A003B274 both showed specific binding to SupT1-CD9B441-HL, but not to the negative control SupT1-CD9B337-HL.

TABLE 2
mAbs A003B192 and A003B274 were screened
for binding to scFv-transfected SupT1 cells.

| | MFI (AF488) | | MFI (AF488) | |
|---|---|---|---|---|
| | SupT1-CD9B441-HL | | SupT1-CD9B337-HL | |
| Protein AA | Std | Staining in | Std | Staining in |
| Name | Staining | Serum | Staining | Serum |
| A003B192 | 62065 | 51535 | 6996 | 9344 |
| A003B274 | 65808 | 73700 | 9853 | 18115 |

### Example 4: Characterization of CD79b CAR (CD9B441-HL) Anti-Idiotypic Antibodies A003B192 and A003B274

Antibodies to detect CD79b CAR (CD9B441-HL) expressed on NK and T cells were identified from panels of proteins derived from Phage Display screening. The proteins were tested initially for binding to recombinant CAR protein and potential binders were scaled up. The proteins were purified and tested for dose dependent binding to SupT1 cells expressing CD9B441-HL by flow cytometry. The binding was determined to be specific to CAR through competition binding experiments with Fc-CD9B441-HL fusion proteins and through lack of binding to parental SupT1 cells. After selection of the best binder, the antibodies were directly conjugated to rPE (recombinant phycoerythrin) for use as CAR detection reagents. The antibodies were purified to contain 1:1 PE:antibody ratio to enable receptor enumeration studies (number of CAR expressed on the cell surface).

Purification

Cell culture supernatant was loaded to MabSelect column and eluted with low pH buffer such as 100 mM sodium acetate pH 3.0, subsequently buffer exchanged to 1×SSC, 8.5% sucrose pH 7.0 using Sephadex G-25 column. Fractions containing protein were collected. Following purification, proteins underwent QC using SDS-PAGE, SEC-HPLC and LC-MS methods.

Phycoerythrin Labeling

1 μL of Modifier reagent from Lightning-Link® R-PE Antibody Labeling Kit (Expedeon cat #703-0015) was added to each 10 μL of antibody to be labeled and mixed gently. The antibody sample (with added Modifier reagent) was pipetted directly onto the lyophilized rPE (Expedeon cat #703-0015), then resuspended gently and incubated for 1 hour in the dark at room temperature (20-25° C.). 1 μL of Quencher reagent was added for every 10 μL of antibody used and incubated for 30 minutes.

After labeling, PE-antibody conjugates were purified on size-exclusion chromatogram column. Fractions were collected and analyzed on SEC-HPLC. Fractions which only contained one antibody with one PE were pooled together and concentrated if necessary. Final products were analyzed on SEC-HPLC.

Characterization of CD79b CAR (CD9B441-HL) Anti-Idiotypic Antibodies A003B192 and A003B274

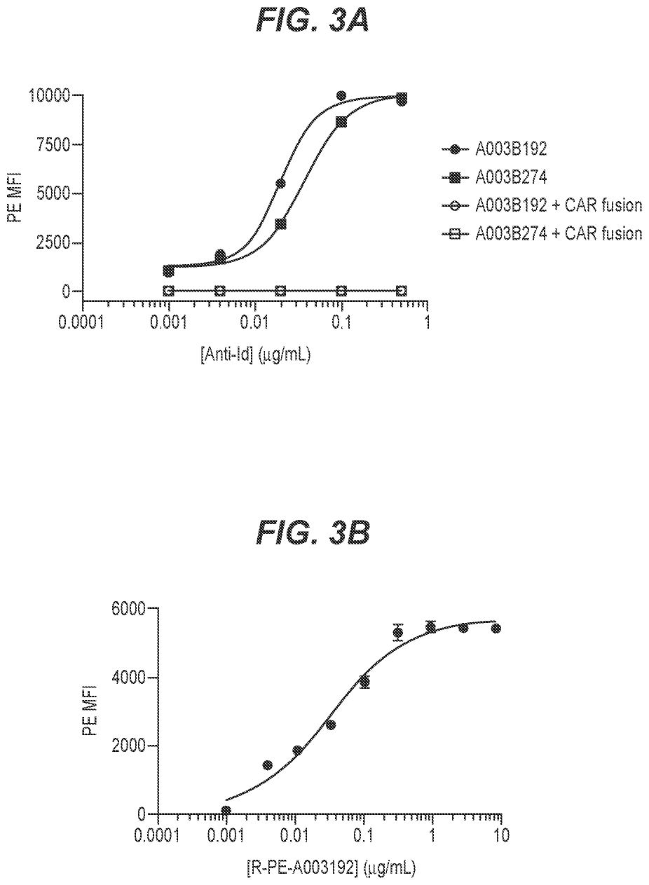

SupT1 cells expressing CD9B441-HL were compared to parental CAR-SupT1 cells. Cells (200,000 cells/well) were stained with LIVE/DEAD Fixable Near-IR viability dye (Life Technologies cat #L10119) and then incubated with increasing concentrations of A003B192 and A003B274 for 45 minutes on ice. In addition, both anti-idiotypic antibodies were tested in the presence of Fc-CD9B441-HL fusion protein to assess the specificity of A003B192 and A003B274 binding to CD9B441-HL CAR. Following incubation, the samples were washed with BSA stain buffer (BD Biosciences cat #554657) and stained with PE-goat anti-mouse IgG polyclonal antibody (Biolegend cat #405307) to detect bound antibody on live CAR+SupT1 cells. After incubation, washing, and fixation (Cytofix, BD Biosciences cat #554655), the samples were acquired on a 10 color FACSCanto II (BD Biosciences) flow cytometer. Analysis was done using FlowJo (TreeStar) and the PE median fluorescent intensity of live CAR+SupT1 cells plotted in FIG. 3A. No binding was detected on CAR-SupT1 parental cells (data not shown) or in the presence of excess Fc-CD9B441-HL CAR fusion protein.

A003B192 was conjugated to rPE and tested for binding to CD9B441-HLSupT1 as described above to ensure binding was not affected by PE labeling. Analysis was done using FlowJo analysis software, and the PE median fluorescent intensity of live CAR+SupT1 cells plotted in FIG. 3B. As shown in FIG. 3B, there was dose-dependent binding of A003B192 to CD9B441-HL-SupT1 cells.

### Embodiments

Particular embodiments of the invention are set forth in the following numbered paragraphs:
1. An anti-idiotype antibody or antigen binding portion thereof that specifically binds a target antibody that comprises CD9B441.
2. The anti-idiotype antibody or antigen-binding portion of paragraph 1, wherein the target antibody or antigen-binding portion thereof comprises a light chain variable (VL) domain having the amino acid sequence of SEQ ID NO: 29 and a heavy chain variable (VH) domain having the amino acid sequence of SEQ ID NO: 30.
3. An anti-idiotype antibody or antigen-binding portion thereof that specifically binds to CD9B441, wherein the anti-idiotype antibody or antigen-binding portion comprises:
(a) a complementarity determining region of a light chain variable (VL) domain comprising,

(i) an LCDR1 having the amino acid sequence of SEQ ID NO: 5;

(ii) an LCDR2 having the amino acid sequence of SEQ ID NO: 6;

(iii) an LCDR3 having the amino acid sequence of SEQ ID NO: 7; and further comprises:

(b) a complementarity determining region of a heavy chain variable (VH) domain comprising HCDR1-3 having amino acid sequences selected from the group consisting of,

(i) an HCDR1 having the amino acid sequence of SEQ ID NO: 17, an HCDR2 having the amino acid sequence of SEQ ID NO: 19, an HCDR3 having the amino acid sequence of SEQ ID NO: 21, and

(ii) an HCDR1 having the amino acid sequence of SEQ ID NO: 18, an HCDR2 having the amino acid sequence of SEQ ID NO: 20, and HCDR3 having the amino acid sequence of SEQ ID NO: 22.

4. The anti-idiotype antibody or antigen-binding portion of paragraph 1 or 3, wherein the VL domain has an amino acid sequence having at least 90% sequence identity to SEQ ID NO: 8.
5. The anti-idiotype antibody or antigen binding portion of paragraph 1 or 3, wherein the light chain has an amino acid sequence having at least 90% sequence identity to SEQ ID NO: 9.
6. An anti-idiotype antibody or antigen-binding portion thereof that specifically binds to a target antibody that comprises CD9B441, wherein the anti-idiotype antibody or antigen-binding portion comprising:
(a) a light chain variable (VL) domain of SEQ ID NO: 8, and
(b) a framework region of a heavy chain variable (VH) domain comprising,

(i) an HFW1 of SEQ ID NO: 11

(ii) an HFW2 having the amino acid sequence selected from the group consisting of SEQ IQ NOs: 12-13

(iii) an HFW3 having the amino acid sequence selected from the group consisting of SEQ ID NOs: 14-15

(iv) an HFW4 of SEQ ID NO: 16

(c) a complementarity determining region of a heavy chain variable (VH) domain comprising,

(i) an HCDR1 having the amino acid sequence selected from the group consisting of SEQ ID NOs: 17-18

(ii) an HCDR2 having the amino acid sequence selected from the group consisting of SEQ ID NOs: 19-20

(iii) an HCDR3 having the amino acid sequence selected from the group consisting of SEQ ID NOs: 21-22.

7. The anti-idiotype antibody or antigen-binding portion of any one of paragraphs 1, 3, and 6; wherein the VH domain has an amino acid sequence having at least 90% sequence identity to an amino acid sequence selected from the group consisting of SEQ ID NOs: 23-24.
8. The anti-idiotype antibody or antigen-binding portion of any one of paragraphs 1, 3, and 6; wherein the heavy chain has an amino acid sequence having at least 90% sequence identity to an amino acid sequence selected from the group consisting of SEQ ID NOs: 25-26.
9. The anti-idiotype antibody or antigen-binding portion of any one of paragraphs 1, 3, and 6; wherein the anti-idiotype antibody or antigen-binding portion comprises the VL domain of SEQ ID NO: 8, and further comprises a VH domain selected from the group consisting of SEQ ID NOs: 23-24.
10. The anti-idiotype antibody or antigen-binding portion of any one of paragraphs 1, 3, and 6; wherein the anti-idiotype antibody or antigen-binding portion comprises a light chain comprising the amino acid sequence of SEQ ID NO: 9 and further comprises a heavy chain comprising an amino acid sequence selected from the group consisting of SEQ ID NOs: 25-26.
11. The anti-idiotype antibody or antigen-binding portion of any one of paragraphs 1, 3, and 6; wherein the antigen binding portion is selected from a Fab, F(ab′)₂, or scFv.
12. The anti-idiotype antibody or antigen-binding portion of any one of paragraphs 1, 3, and 6; wherein the antibody is a monoclonal antibody.
13. The anti-idiotype antibody or antigen-binding portion of any one of paragraphs 1, 3, and 6; wherein the antibody is a chimeric antibody.
14. The anti-idiotype antibody or antigen-binding portion of any one of paragraphs 1, 3, and 6; wherein the antibody comprises a murine IgG2a framework.
15. The anti-idiotype antibody or antigen-binding portion of any one of paragraphs 1, 3, and 6; wherein the antibody is a fully human antibody.
16. A nucleic acid encoding the heavy chain, the light chain, or both, of the anti-idiotype antibody or antigen-binding portion of any one of paragraphs 1, 3, and 6.
17. A nucleic acid encoding the heavy chain, the light chain, or both, of an anti-idiotype antibody or an antigen-binding portion thereof that specifically binds CD9B441, wherein said nucleic acid comprises:
a) the nucleotide sequence of SEQ ID NO: 10;
b) the nucleotide sequence selected from the group consisting of SEQ ID NOs: 27-28; or
c) both a) and b).
18. A vector comprising the nucleic acid of paragraph 17.
19. The vector of paragraph 18, wherein the vector is an expression vector.
20. A host cell comprising the vector of paragraph 19.
21. The host cell of paragraph 20, wherein the cell is a mammalian cell.
22. A method of producing an anti-idiotype antibody or antigen-binding portion thereof that specifically binds CD9B441, said method comprising culturing the host cell of paragraph 20 under conditions that allow said antibody or antigen-binding portion to be expressed, wherein the host cell comprises nucleotide sequences coding the heavy chain and light chain of the antibody or antigen-binding portion, and isolating said antibody or antigen-binding portion from the culture.
23. A method for detecting CD9B441 in a biologic sample comprising: (a) providing a biological sample; (b) contacting the biological sample with the anti-idiotype antibody or antigen-binding portion of any one of paragraphs 1, 3, and 6; and (c) detecting the anti-idiotype antibody or antigen-binding portion.
24. The method according to paragraph 23, wherein the antibody comprises a detectable label.
25. The method according to paragraph 23, wherein the method further comprises contacting the anti-idiotype antibody or antigen-binding portion with a detectable label before detecting the anti-idiotype antibody or antigen-binding portion.
26. The method according to paragraph 23, wherein the biological sample is blood, serum or urine.
27. A method for detecting expression of a chimeric antigen receptor (CAR) comprising CD9B441 in a biologic sample comprising: (a) providing a biological sample; (b) contacting the biological sample with the anti-idiotype antibody or antigen-binding portion of any one of paragraphs 1, 3, and 6; and (c) detecting the anti-idiotype antibody or antigen-binding portion, and thereby detecting the expression of the CAR.
28. The anti-idiotype antibody or antigen-binding portion of any one of paragraphs 1, 3, and 6; wherein CD9B441 is within the antigen-binding domain of the extracellular portion of a chimeric antigen receptor (CAR).
29. The anti-idiotype antibody or antigen-binding portion of paragraph 28 wherein CD9B441 is an scFv, and the anti-idiotype antibody or antigen-binding portion specifically binds an epitope in the scFv of the CAR.
30. The anti-idiotype antibody or antigen-binding portion of paragraph 28, wherein CD9B441 specifically binds CD79b.
31. The anti-idiotype antibody or antigen-binding portion of paragraph 28, wherein the antibody or antigen-binding portion does not cross-react to other CD79b antibodies or other CD79b-binding CARs.
32. The anti-idiotype antibody or antigen-binding portion of paragraph 28, wherein the CAR has an amino acid sequence selected from the group consisting of SEQ ID NO: 31-32.
33. A kit for detecting CD9B441 in a biologic sample comprising: (a) the anti-idiotype antibody or antigen-binding portion of any one of paragraphs 1, 3, and 6; and (b) instructions for detecting the anti-idiotype antibody or antigen-binding portion.
34. The anti-idiotype antibody or antigen-binding portion of any one of paragraphs 1, 3, and 6; for use in detecting CD9B441 in a biologic sample comprising: (a) providing a biological sample; (b) contacting the biological sample with the anti-idiotype antibody or antigen-binding portion; and (c) detecting the anti-idiotype antibody or antigen-binding portion.
35. A method of purifying CD9B441 from a sample comprising: (a) providing a biological sample comprising CD9B441; (b) contacting the biological sample with the anti-idiotype antibody or antigen-binding portion of any one of paragraphs 1, 3, and 6; and (c) capturing the anti-idiotype antibody or antigen-binding portion, and thereby purifying CD9B441.
36. A method of selecting CAR-T cells from a cell population comprising: (a) providing a biological sample comprising CAR-T cells; (b) contacting the biological sample with the anti-idiotype antibody or antigen-binding portion of any one of paragraphs 1, 3, and 6; and (c) capturing the anti-idiotype antibody or antigen-binding portion, and thereby selecting CAR-T cells.

### INCORPORATION BY REFERENCE

All publications and patents mentioned herein are hereby incorporated by reference in their entirety as if each individual publication or patent was specifically and individually indicated to be incorporated by reference.

While specific embodiments of the subject disclosure have been discussed, the above specification is illustrative and not restrictive. Many variations of the disclosure will become apparent to those skilled in the art upon review of this specification and the claims below. The full scope of the disclosure should be determined by reference to the claims, along with their full scope of equivalents, and the specification, along with such variations

SEQUENCES
SEQ ID No. 1:
LFWI (amino acid)
EIVLTQSPGTLSLSPGERATLSCRAS
SEQ ID No. 2:
LFW2 (amino acid)
LAWYQQKPGQAPRLLIY
SEQ ID No. 3:
LFW3 (amino acid)
SRATGIPDRFSGSGSGTDFTLTISRLEPEDFAVYYC
SEQ ID No. 4:
LFW4 (amino acid)
FGQGTKVEIK
SEQ ID No. 5:
LCDR1 (amino acid)
QSVSSSY
SEQ ID No. 6:
LCDR2 (amino acid)
GAS
SEQ ID No. 7:
LCDR3 (amino acid)
QQYGSSPLT
SEQ ID No. 8:
Light chain variable (VL) domain
(amino acid)
EIVLTQSPGTLSLSPGERATLSCRASQSVSSSYLA
WYQQKPGQAPRLLIYGASSRATGIPDRFSGSGSGT
DFTLTISRLEPEDFAVYYCQQYGSSPLTFGQGTKV
EIK
SEQ ID No. 9:
Light chain (amino acid)
EIVLTQSPGTLSLSPGERATLSCRASQSVSSSYLA
WYQQKPGQAPRLLIYGASSRATGIPDRFSGSGSGT
DFTLTISRLEPEDFAVYYCQQYGSSPLTFGQGTKV
EIKRADAAPTVSIFPPSSEQLTSGGASVVCFLNNF
YPKDINVKWKIDGSERQNGVLNSWTDQDSKDSTYS
MSSTLTLTKDEYERHNSYTCEATHKTSTSPIVKSF
NRNEC
SEQ ID No. 10:
Light chain (DNA)
GAAATTGTGCTGACCCAGAGCCCGGGCACCCTGAG
CCTGAGCCCGGGCGAACGCGCGACCCTGAGCTGCC
GCGCGAGCCAGAGCGTGAGCAGCAGCTATCTGGCG
TGGTATCAGCAGAAACCGGGCCAGGCGCCGCGCCT
GCTGATTTATGGCGCGAGCAGCCGCGCGACCGGCA
TTCCGGATCGCTTTAGCGGCAGCGGTTCCGGCACC
GATTTTACCCTGACCATTAGCCGCCTGGAACCGGA
AGATTTTGCGGTGTATTATTGCCAGCAGTATGGCA
GCAGCCCGCTGACCTTTGGCCAGGGCACCAAAGTG
GAAATTAAACGGGCTGATGCTGCACCGACTGTGTC
CATCTTCCCACCATCCAGTGAGCAGTTAACATCTG
GAGGTGCCTCAGTCGTGTGCTTCTTGAACAACTTC
TACCCCAAAGACATCAATGTCAAGTGGAAGATTGA
TGGCAGTGAACGACAAAATGGCGTCCTGAACAGTT
GGACTGATCAGGACAGCAAAGACAGCACCTACAGC
ATGAGCAGCACCCTCACGTTGACCAAGGACGAGTA
TGAACGACATAACAGCTATACCTGTGAGGCCACTC
ACAAGACATCAACTTCACCCATTGTCAAGAGCTTC
AACAGGAATGAGTGT
SEQ ID No. 11:
HFW1 (amino acid)
QVQLVQSGAEVKKPGSSVKVSCKAS
SEQ ID No. 12:
HFW2 of A003B192 (amino acid)
ISWVRQAPGQGLEWMGG
SEQ ID No. 13:
HFW2 of A003B274 (amino acid)
ISWVRQAPGQGLEWMGY
SEQ ID No. 14:
HFW3 of A003B192 (amino acid)
EYAQKFQGRVTITADESTSTAYMELSSLRSEDTA
VYYC
SEQ ID No. 15:
HFW3 of A003B274 (amino acid)
NYAQKFQGRVTITADESTSTAYMELSSLRSEDTA
VYYC
SEQ ID No. 16:
HFW4 (amino acid)
WGQGTLVTVSS
SEQ ID No. 17:
HCDR1 of A003B192 (amino acid)
GGTFKSDA
SEQ ID No. 18:
HCDR1 of A003B274 (amino acid)
GGTFKSYA
SEQ ID No. 19:
HCDR2 of A003B192 (amino acid)
IRPNEGNA
SEQ ID No. 20:
HCDR2 of A003B274 (amino acid)
ISPESGTA
SEQ ID No. 21:
HCDR3 of A003B192 (amino acid)
ARGRYGAYRLVYYAFDY
SEQ ID No. 22:
HCDR3 of A003B274 (amino acid)
ARERYYYGYRRYRYYGMDV
SEQ ID No. 23:
Heavy chain variable (VH) domain
of A003B192 (amino acid)
QVQLVQSGAEVKKPGSSVKVSCKASGGTFKSDAIS
WVRQAPGQGLEWMGGIRPNEGNAEYAQKFQGRVTI
TADESTSTAYMELSSLRSEDTAVYYCARGRYGAYR
LVYYAFDYWGQGTLVTVSS
SEQ ID No. 24:
Heavy chain variable (VH) domain of
A003B274 (amino acid)
QVQLVQSGAEVKKPGSSVKVSCKASGGTFKSYAIS
WVRQAPGQGLEWMGYISPESGTANYAQKFQGRVTI
TADESTSTAYMELSSLRSEDTAVYYCARERYYYGY
RRYRYYGMDVWGQGTLVTVSS
SEQ ID No. 25:
Heavy chain of A003B192 (amino acid)
QVQLVQSGAEVKKPGSSVKVSCKASGGTFKSDAIS
WVRQAPGQGLEWMGGIRPNEGNAEYAQKFQGRVTI
TADESTSTAYMELSSLRSEDTAVYYCARGRYGAYR
LVYYAFDYWGQGTLVTVSSAKTTAPSVYPLAPVCG
DTTGSSVTLGCLVKGYFPEPVTLTWNSGSLSSGVH
TFPAVLQSDLYTLSSSVTVTSSTWPSQSITCNVAH
PASSTKVDKKIEPRGPTIKPCPPCKCPAPNLLGGP
SVFIFPPKIKDVLMISLSPIVTCVVVDVSEDDPDV
QISWFVNNVEVHTAQTQTHREDYNSTLRVVSALPI
QHQDWMSGKEFKCKVNNKDLPAPIERTISKPKGSV
RAPQVYVLPPPEEEMTKKQVTLTCMVTDFMPEDIY
VEWTNNGKTELNYKNTEPVLDSDGSYFMYSKLRVE
KKNWVERNSYSCSVVHEGLHNHHTTKSFSRTPGK
SEQ ID No. 26:
Heavy chain of A003B274 (amino acid)
QVQLVQSGAEVKKPGSSVKVSCKASGGTFKSYAIS
WVRQAPGQGLEWMGYISPESGTANYAQKFQGRVTI
TADESTSTAYMELSSLRSEDTAVYYCARERYYYGY
RRYRYYGMDVWGQGTLVTVSSAKTTAPSVYPLAPV
CGDTTGSSVTLGCLVKGYFPEPVTLTWNSGSLSSG
VHTFPAVLQSDLYTLSSSVTVTSSTWPSQSITCNV
AHPASSTKVDKKIEPRGPTIKPCPPCKCPAPNLLG
GPSVFIFPPKIKDVLMISLSPIVTCVVVDVSEDDP
DVQISWFVNNVEVHTAQTQTHREDYNSTLRVVSAL
PIQHQDWMSGKEFKCKVNNKDLPAPIERTISKPKG
SVRAPQVYVLPPPEEEMTKKQVTLTCMVTDFMPED
IYVEWTNNGKTELNYKNTEPVLDSDGSYFMYSKLR
VEKKNWVERNSYSCSVVHEGLHNHHTTKSFSRTPG
K
SEQ ID No. 27:
Heavy chain of A003B192 (DNA)
CAGGTGCAGCTGGTGCAGAGCGGCGCGGAAGTGAA
AAAACCGGGCAGCAGCGTGAAAGTGAGCTGCAAAG
CGAGCGGCGGCACCTTCAAATCCGACGCGATTAGC
TGGGTGCGCCAGGCGCCGGGCCAGGGCCTGGAATG
GATGGGCGGTATTCGCCCAAACGAGGGGAATGCTG
AGTACGCGCAGAAATTTCAGGGCCGCGTGACCATT
ACCGCTGATGAAAGCACCAGCACCGCGTATATGGA
ACTGAGCAGCCTGCGCAGCGAAGATACCGCGGTGT
ATTATTGCGCGCGTGGTCGATATGGTGCATATCGT
CTGGTTTACTATGCGTTTGACTACTGGGGCCAGGG
CACCCTGGTGACCGTCTCGAGTGCCAAAACAACAG
CACCAAGTGTCTATCCACTGGCCCCTGTGTGTGGA
GATACAACTGGCTCCTCGGTGACTCTAGGATGCCT
GGTCAAGGGTTATTTCCCTGAGCCAGTGACCTTGA
CCTGGAACTCTGGATCCCTGTCCAGTGGTGTGCAC
ACCTTCCCAGCTGTCCTGCAGTCTGACCTCTACAC
CCTCAGCAGCTCAGTGACTGTAACCTCGAGCACCT
GGCCCAGCCAGTCCATCACCTGCAATGTGGCCCAC
CCGGCAAGCAGCACCAAGGTGGACAAGAAAATTGA
GCCCAGAGGGCCCACAATCAAGCCCTGTCCTCCAT
GCAAATGCCCAGCACCTAACCTCTTGGGTGGACCA
TCCGTCTTCATCTTCCCTCCAAAGATCAAGGATGT
ACTCATGATCTCCCTGAGCCCCATAGTCACATGTG
TGGTGGTGGATGTGAGCGAGGATGACCCAGATGTC
CAGATCAGCTGGTTTGTGAACAACGTGGAAGTACA
CACAGCTCAGACACAAACCCATAGAGAGGATTACA
ACAGTACTCTCCGGGTGGTCAGTGCCCTCCCCATC
CAGCACCAGGACTGGATGAGTGGCAAGGAGTTCAA
ATGCAAGGTCAACAACAAAGACCTCCCAGCGCCCA
TCGAGAGAACCATCTCAAAACCCAAAGGGTCAGTA
AGAGCTCCACAGGTATATGTCTTGCCTCCACCAGA
AGAAGAGATGACTAAGAAACAGGTCACTCTGACCT
GCATGGTCACAGACTTCATGCCTGAAGACATTTAC
GTGGAGTGGACCAACAACGGGAAAACAGAGCTAAA
CTACAAGAACACTGAACCAGTCCTGGACTCTGATG
GTTCTTACTTCATGTACAGCAAGCTGAGAGTGGAA
AAGAAGAACTGGGTGGAAAGAAATAGCTACTCCTG
TTCAGTGGTCCACGAGGGTCTGCACAATCACCACA
CGACTAAGAGCTTCTCCCGGACTCCGGGTAAA
SEQ ID No. 28:
Heavy chain of A003B274 (DNA)
CAGGTGCAGCTGGTGCAGAGCGGCGCGGAAGTGAA
AAAACCGGGCAGCAGCGTGAAAGTGAGCTGCAAAG
CGAGCGGCGGCACCTTTAAATCCTATGCGATTTCC
TGGGTGCGCCAGGCGCCGGGCCAGGGCCTGGAATG
GATGGGCTATATTTCCCCAGAGAGTGGCACTGCCA
ATTATGCGCAGAAATTTCAGGGCCGCGTGACCATT
ACCGCTGATGAAAGCACCAGCACCGCGTATATGGA
ACTGAGCAGCCTGCGCAGCGAAGATACCGCGGTGT
ATTATTGCGCGCGTGAACGTTACTACTATGGCTAT
CGTCGTTACCGGTATTACGGTATGGATGTTTGGGG
CCAGGGCACCCTGGTGACCGTCTCGAGTGCCAAAA
CAACAGCACCAAGTGTCTATCCACTGGCCCCTGTG
TGTGGAGATACAACTGGCTCCTCGGTGACTCTAGG
ATGCCTGGTCAAGGGTTATTTCCCTGAGCCAGTGA
CCTTGACCTGGAACTCTGGATCCCTGTCCAGTGGT
GTGCACACCTTCCCAGCTGTCCTGCAGTCTGACCT
CTACACCCTCAGCAGCTCAGTGACTGTAACCTCGA
GCACCTGGCCCAGCCAGTCCATCACCTGCAATGTG
GCCCACCCGGCAAGCAGCACCAAGGTGGACAAGAA
AATTGAGCCCAGAGGGCCCACAATCAAGCCCTGTC
CTCCATGCAAATGCCCAGCACCTAACCTCTTGGGT
GGACCATCCGTCTTCATCTTCCCTCCAAAGATCAA
GGATGTACTCATGATCTCCCTGAGCCCCATAGTCA
CATGTGTGGTGGTGGATGTGAGCGAGGATGACCCA
GATGTCCAGATCAGCTGGTTTGTGAACAACGTGGA
AGTACACACAGCTCAGACACAAACCCATAGAGAGG
ATTACAACAGTACTCTCCGGGTGGTCAGTGCCCTC
CCCATCCAGCACCAGGACTGGATGAGTGGCAAGGA
GTTCAAATGCAAGGTCAACAACAAAGACCTCCCAG
CGCCCATCGAGAGAACCATCTCAAAACCCAAAGGG
TCAGTAAGAGCTCCACAGGTATATGTCTTGCCTCC
ACCAGAAGAAGAGATGACTAAGAAACAGGTCACTC
TGACCTGCATGGTCACAGACTTCATGCCTGAAGAC
ATTTACGTGGAGTGGACCAACAACGGGAAAACAGA
GCTAAACTACAAGAACACTGAACCAGTCCTGGACT
CTGATGGTTCTTACTTCATGTACAGCAAGCTGAGA
GTGGAAAAGAAGAACTGGGTGGAAAGAAATAGCTA
CTCCTGTTCAGTGGTCCACGAGGGTCTGCACAATC
ACCACACGACTAAGAGCTTCTCCCGGACTCCGGGT
AAA
SEQ ID No. 29:
Light chain variable (VL)
of CD9B441 (amino acid)
QSALTQPPSVSEAPRQRVTISCSGSASNIGNNGVN
WYQQLPGKTPKLLIYNDDLLPSGVSDRFSGSKSGT
SASLAISGLQSEDEADYFCAAWDDSLNGLVFGGGT
KLTVL
SEQ ID No. 30:
Heavy chain variable (VH) of
CD9B441 (amino acid)
QVQLQQSGPGLVKPSQTLSLTCAISGDSVSSKSGA
WNWIRQSPSRGLEWLGRTYYRSKWYNEYAVSVKSR
ITINPDTSKNQFSLQLNSVTPEDTAVYYCTRVDTD
FDYWGQGTLVTVSS
SEQ ID No. 31:
CAR 1 (CD9B441-LH) (amino acid)
MAWVWTLLFLMAAAQSIQAQSALTQPPSVSEAPRQ
RVTISCSGSASNIGNNGVNWYQQLPGKTPKLLIYN
DDLLPSGVSDRFSGSKSGTSASLAISGLQSEDEAD
YFCAAWDDSLNGLVFGGGTKLTVLGGSEGKSSGSG
SESKSTGGSQVQLQQSGPGLVKPSQTLSLTCAISG
DSVSSKSGAWNWIRQSPSRGLEWLGRTYYRSKWYN
EYAVSVKSRITINPDTSKNQFSLQLNSVTPEDTAV
YYCTRVDTDFDYWGQGTLVTVSSTSTPAPRPPTPA
PTIASQPLSLRPEACRPAAGGAVHTRGLDFACDIY
IWAPLAGTCGVLLLSLVITLYCKRGRKKLLYIFKQ
PFMRPVQTTQEEDGCSCRFPEEEEGGCELRVKFSR
SADAPAYKQGQNQLYNELNLGRREEYDVLDKRRGR
DPEMG
GKPRRKNPQEGLYNELQKDKMAEAYSEIGMKGERR
RGKGHDGLYQGLSTATKDTYDALHMQALPPR
SEQ ID No. 32:
CAR 2 (CD9B441-HL) (amino acid)
MAWVWTLLFLMAAAQSIQAQVQLQQSGPGLVKPSQ
TLSLTCAISGDSVSSKSGAWNWIRQSPSRGLEWLG
RTYYRSKWYNEYAVSVKSRITINPDTSKNQFSLQL
NSVTPEDTAVYYCTRVDTDFDYWGQGTLVTVSSGG
SEGKSSGSGSESKSTGGSQSALTQPPSVSEAPRQR
VTISCSGSASNIGNNGVNWYQQLPGKTPKLLIYND
DLLPSGVSDRFSGSKSGTSASLAISGLQSEDEADY
FCAAWDDSLNGLVFGGGTKLTVLTSTPAPRPPTPA
PTIASQPLSLRPEACRPAAGGAVHTRGLDFACDIY
IWAPLAGTCGVLLLSLVITLYCKRGRKKLLYIFKQ
PFMRPVQTTQEEDGCSCRFPEEEEGGCELRVKFSR
SADAPAYKQGQNQLYNELNLGRREEYDVLDKRRGR
DPEMGGKPRRKNPQEGLYNELQKDKMAEAYSEIGM
KGERRRGKGHDGLYQGLSTATKDTYDALHMQALPP
R
SEQ ID No. 33:
human CD79b sequence (amino acid)
MARLALSPVPSHWMVALLLLLSAEPVPAARSEDRY
RNPKGSACSRIWQSPRFIARKRGFTVKMHCYMNSA
SGNVSWLWKQEMDENPQQLKLEKGRMEESQNESLA
TLTIQGIRFEDNGIYFCQQKCNNTSEVYQGCGTEL
RVMGFSTLAQLKQRNTLKDGIIMIQTLLIILFIIV
PIFLLLDKDDSKAGMEEDHTYEGLDIDQTATYEDI
VTLRTGEVKWSVGEHPGQE

We claim:

1. An anti-idiotype antibody or antigen binding portion thereof that specifically binds a target antibody that comprises CD9B441.

2. The anti-idiotype antibody or antigen-binding portion of claim 1, wherein the target antibody or antigen-binding portion thereof comprises a light chain variable (VL) domain having the amino acid sequence of SEQ ID NO: 29 and a heavy chain variable (VH) domain having the amino acid sequence of SEQ ID NO: 30.

3. An anti-idiotype antibody or antigen-binding portion thereof that specifically binds to CD9B441, wherein the anti-idiotype antibody or antigen-binding portion comprises:
(a) a complementarity determining region of a light chain variable (VL) domain comprising,
(i) an LCDR1 having the amino acid sequence of SEQ ID NO: 5;
(ii) an LCDR2 having the amino acid sequence of SEQ ID NO: 6;
(iii) an LCDR3 having the amino acid sequence of SEQ ID NO: 7; and further comprises:
(b) a complementarity determining region of a heavy chain variable (VH) domain comprising HCDR1-3 having amino acid sequences selected from the group consisting of,
(i) an HCDR1 having the amino acid sequence of SEQ ID NO: 17, an HCDR2 having the amino acid sequence of SEQ ID NO: 19, an HCDR3 having the amino acid sequence of SEQ ID NO: 21, and
(ii) an HCDR1 having the amino acid sequence of SEQ ID NO: 18, an HCDR2 having the amino acid sequence of SEQ ID NO: 20, and HCDR3 having the amino acid sequence of SEQ ID NO: 22.

4. The anti-idiotype antibody or antigen-binding portion of claim 1 or 3, wherein the VL domain has an amino acid sequence having at least 90% sequence identity to SEQ ID NO: 8.

5. The anti-idiotype antibody or antigen binding portion of claim 1 or 3, wherein the light chain has an amino acid sequence having at least 90% sequence identity to SEQ ID NO: 9.

6. An anti-idiotype antibody or antigen-binding portion thereof that specifically binds to a target antibody that comprises CD9B441, wherein the anti-idiotype antibody or antigen-binding portion comprising:
(a) a light chain variable (VL) domain of SEQ ID NO: 8, and
(b) a framework region of a heavy chain variable (VH) domain comprising,
(i) an HFW1 of SEQ ID NO: 11
(ii) an HFW2 having the amino acid sequence selected from the group consisting of SEQ IQ NOs: 12-13
(iii) an HFW3 having the amino acid sequence selected from the group consisting of SEQ ID NOs: 14-15
(iv) an HFW4 of SEQ ID NO: 16
(c) a complementarity determining region of a heavy chain variable (VH) domain comprising,
(i) an HCDR1 having the amino acid sequence selected from the group consisting of SEQ ID NOs: 17-18
(ii) an HCDR2 having the amino acid sequence selected from the group consisting of SEQ ID NOs: 19-20
(iii) an HCDR3 having the amino acid sequence selected from the group consisting of SEQ ID NOs: 21-22.

7. The anti-idiotype antibody or antigen-binding portion of any one of claims 1, 3, and 6; wherein the VH domain has an amino acid sequence having at least 90% sequence identity to an amino acid sequence selected from the group consisting of SEQ ID NOs: 23-24.

8. The anti-idiotype antibody or antigen-binding portion of any one of claims 1, 3, and 6; wherein the heavy chain has an amino acid sequence having at least 90% sequence identity to an amino acid sequence selected from the group consisting of SEQ ID NOs: 25-26.

9. The anti-idiotype antibody or antigen-binding portion of any one of claims 1, 3, and 6; wherein the anti-idiotype antibody or antigen-binding portion comprises the VL domain of SEQ ID NO: 8, and further comprises a VH domain selected from the group consisting of SEQ ID NOs: 23-24.

10. The anti-idiotype antibody or antigen-binding portion of any one of claims 1, 3, and 6; wherein the anti-idiotype antibody or antigen-binding portion comprises a light chain comprising the amino acid sequence of SEQ ID NO: 9 and further comprises a heavy chain comprising an amino acid sequence selected from the group consisting of SEQ ID NOs: 25-26.

11. The anti-idiotype antibody or antigen-binding portion of any one of claims 1, 3, and 6; wherein the antigen binding portion is selected from a Fab, F(ab′)₂, or scFv.

12. The anti-idiotype antibody or antigen-binding portion of any one of claims 1, 3, and 6; wherein the antibody is a monoclonal antibody.

13. The anti-idiotype antibody or antigen-binding portion of any one of claims 1, 3, and 6; wherein the antibody is a chimeric antibody.

14. The anti-idiotype antibody or antigen-binding portion of any one of claims 1, 3, and 6; wherein the antibody comprises a murine IgG2a framework.

15. The anti-idiotype antibody or antigen-binding portion of any one of claims 1, 3, and 6; wherein the antibody is a fully human antibody.

16. A nucleic acid encoding the heavy chain, the light chain, or both, of the anti-idiotype antibody or antigen-binding portion of any one of claims 1, 3, and 6.

17. A nucleic acid encoding the heavy chain, the light chain, or both, of an anti-idiotype antibody or an antigen-binding portion thereof that specifically binds CD9B441, wherein said nucleic acid comprises:
a) the nucleotide sequence of SEQ ID NO: 10;
b) the nucleotide sequence selected from the group consisting of SEQ ID NOs: 27-28; or
c) both a) and b).

18. A vector comprising the nucleic acid of claim 17.

19. The vector of claim 18, wherein the vector is an expression vector.

20. A host cell comprising the vector of claim 19.

21. The host cell of claim 20, wherein the cell is a mammalian cell.

22. A method of producing an anti-idiotype antibody or antigen-binding portion thereof that specifically binds CD9B441, said method comprising culturing the host cell of claim 20 under conditions that allow said antibody or antigen-binding portion to be expressed, wherein the host cell comprises nucleotide sequences coding the heavy chain and light chain of the antibody or antigen-binding portion, and isolating said antibody or antigen-binding portion from the culture.

23. A method for detecting CD9B441 in a biologic sample comprising: (a) providing a biological sample; (b) contacting the biological sample with the anti-idiotype antibody or antigen-binding portion of any one of claims 1, 3, and 6; and (c) detecting the anti-idiotype antibody or antigen-binding portion.

24. The method according to claim 23, wherein the antibody comprises a detectable label.

25. The method according to claim 23, wherein the method further comprises contacting the anti-idiotype antibody or antigen-binding portion with a detectable label before detecting the anti-idiotype antibody or antigen-binding portion.

26. The method according to claim 23, wherein the biological sample is blood, serum or urine.

27. A method for detecting expression of a chimeric antigen receptor (CAR) comprising CD9B441 in a biologic sample comprising: (a) providing a biological sample; (b) contacting the biological sample with the anti-idiotype antibody or antigen-binding portion of any one of claims 1, 3, and 6; and (c) detecting the anti-idiotype antibody or antigen-binding portion, and thereby detecting the expression of the CAR.

28. The anti-idiotype antibody or antigen-binding portion of any one of claims 1, 3, and 6; wherein CD9B441 is within the antigen-binding domain of the extracellular portion of a chimeric antigen receptor (CAR).

29. The anti-idiotype antibody or antigen-binding portion of claim 28 wherein CD9B441 is an scFv, and the anti-idiotype antibody or antigen-binding portion specifically binds an epitope in the scFv of the CAR.

30. The anti-idiotype antibody or antigen-binding portion of claim 28, wherein CD9B441 specifically binds CD79b.

31. The anti-idiotype antibody or antigen-binding portion of claim 28, wherein the antibody or antigen-binding portion does not cross-react to other CD79b antibodies or other CD79b-binding CARs.

32. The anti-idiotype antibody or antigen-binding portion of claim 28, wherein the CAR has an amino acid sequence selected from the group consisting of SEQ ID NO: 31-32.

33. A kit for detecting CD9B441 in a biologic sample comprising: (a) the anti-idiotype antibody or antigen-binding portion of any one of claims 1, 3, and 6; and (b) instructions for detecting the anti-idiotype antibody or antigen-binding portion.

34. The anti-idiotype antibody or antigen-binding portion of any one of claims 1, 3, and 6; for use in detecting CD9B441 in a biologic sample comprising: (a) providing a biological sample; (b) contacting the biological sample with the anti-idiotype antibody or antigen-binding portion; and (c) detecting the anti-idiotype antibody or antigen-binding portion.

35. A method of purifying CD9B441 from a sample comprising: (a) providing a biological sample comprising CD9B441; (b) contacting the biological sample with the anti-idiotype antibody or antigen-binding portion of any one of claims 1, 3, and 6; and (c) capturing the anti-idiotype antibody or antigen-binding portion, and thereby purifying CD9B441.

36. A method of selecting CAR-T cells from a cell population comprising: (a) providing a biological sample comprising CAR-T cells; (b) contacting the biological sample with the anti-idiotype antibody or antigen-binding portion of any one of claims 1, 3, and 6; and (c) capturing the anti-idiotype antibody or antigen-binding portion, and thereby selecting CAR-T cells.
